(19) United States
(12) Patent Application Publication
(10) Pub. No.: US 2017/0342500 A1
(43) Pub. Date: Nov. 30, 2017

(54) METHOD FOR IDENTIFICATION OF TISSUE OR ORGAN LOCALIZATION OF A TUMOUR

(71) Applicant: Danmarks Tekniske Universitet, Kgs. Lyngby (DK)

(72) Inventors: Andrea Marion Marquard, Copenhagen N (DK); Aron Charles Eklund, Copenhagen Ø (DK); Nicolai Juul Birkbak, London (GB); Zoltan Imre Szallasi, Boston, MA (US)

(21) Appl. No.: 15/537,645

(22) PCT Filed: Dec. 18, 2015
(86) PCT No.: PCT/EP2015/080411

§ 371 Date: Jun. 19, 2017

(30) Foreign Application Priority Data
Dec. 19, 2014 (EP) 14199179.4

(51) Int. Cl. C12Q 1/68

(52) U.S. Cl. CPC C12Q 1/6886; C12Q 2600/178; C12Q 2600/112; C12Q 2600/156

(57) ABSTRACT

The invention relates to a method for predicting the localization of a primary tumour, wherein said method comprises the use of genomic profile data, and wherein the method is capable of predicting the type of cancer by a classification score ranking among a variety of the possible tumour types.

### FIELD OF INVENTION

The invention relates to a method for predicting the localization of a primary tumour, wherein said method comprises the use of genomic profile data, and wherein the method is capable of predicting the type of cancer by a classification score ranking among a variety of the possible tumour types.

### BACKGROUND OF INVENTION

Cancer, also known as a malignant tumour, is a widespread disease with several millions of new cases globally each year. More than 8 million people died of cancer worldwide in 2012.

The mortality from cancer can be reduced by an early detection. If the tumour is located early, it can be removed and/or a treatment can be tailored to the specific cancer type.

However, in many cases of new cancer patients, the patients present with metastatic cancer for which the primary tumour (tissue wherein the cancer has started) cannot be readily located.

To initiate curative treatment, it is advantageous to locate the primary tumour. Diagnosis of cancer with unknown primary origin often involves many time consuming and costly clinical tests, since a selection of laboratory or imaging tests has to be made in different tissues in order to localize the primary tumour. Furthermore, in 2-4% of the cases, the primary tumour is never located.

Cancer arises as a result of changes (e.g. mutations) in the genomes of cells within the tissue of the primary origin of cancer, and these changes are reflected in the RNA and proteins produced by a cancer cell, as well as in the regulation of expression of genes in the cancer cell. Thus, cancer cells such as those of a primary tumour, or of a metastatic tumour, hold information regarding which genomic changes has resulted in cancer. It is further known that tumours located in variety of organs shed the mutated DNA into e.g. the bloodstream, also known as circulating tumour DNA (ctDNA), and in some cases tumour DNA is also present in other bodily fluids, such as urine.

Copy number variations (CNV) in genomic regions or genes have also been found previously to be associated with cancer. CNV is alterations of the genomes, reflected as the structural variation in the number of copies of one or more sections of the DNA. The alterations may comprise deletion of sections of the DNA, or duplication of sections of the DNA.

Several different types of genomic changes have been associated with specific cancer types. F. Dietlein and W. Eschner have described a method with the purpose of identifying primary tumour origin based on mutation in specific genes. However, the input data used in this method is restricted to only use mutational information for a gene if it can be unambiguously associated with a single cancer type. The method does not take into account copy number variation in the cancer_{;} nor the frequency of specific types of nucleotide base substitutions regardless of where in the genome they occur.

WO 09105154 describes a method to diagnose cancer types showing gene copy number variations (CNV), for example lymphoma, leukemia, glioma, breast cancer or lung cancer based on expression data from at least 5 nucleic acid sequences encoding proteins from a patient. The method disclosed in WO 09105154 does not involve a method that uses the combination of mutational status in genes mutated in cancer and CNV information. It does also not mention a method wherein the classification scores of different cancer types are ranked.

Beroukhim et al. have studied somatic copy number variation alterations (SCNA) across human cancers, and concluded that most of the significant SCNAs within any single cancer type tend to be found in other cancer types as well.

Increased frequency of specific types of single substitutions have previously been observed and described in the prior art (Alexandrov 2013 a and b, and WO080211288). Alexandrov 2013a discloses that it is possible to derive 20 distinct mutational signatures, wherein some are present in many cancer types. These documents do not disclose the use of such information in combination with mutation status of genes mutated in cancer, or a method wherein the classification scores of different cancer types are ranked and used for prediction.

### SUMMARY OF INVENTION

Considering the prior art described above, it is an object of the present invention to provide a method that can predict the localization of a primary tumour selected among a plurality of cancer types with improved accuracy.

The object can be achieved by a method for prediction of a specific type of cancer in a subject using an acquired bodily sample from said subject, said method comprising the steps of;

a) providing biological sequences derived from said bodily sample,

b) deriving the mutation status of specific genes that are mutated in cancer from said biological sequences compared to a normal sample,

c) calculating one or more of the following types of information i) to iii) from said biological sequences:
i) single base substitution frequency wherein the identity of the two bases flanking said substitution is not taken into account,
ii) single base substitution frequency in triplets of nucleotide bases, wherein the identity of the two bases flanking the substitution is taken into account,
iii) copy number variation (CNV) of genomic regions and/or genes compared to the copy number of the same regions and/or genes in a normal sample

d) calculating a classification score for the presence of each of a plurality of cancer types in said subject, wherein said classification score calculation is based on the mutation status derived from step b) in combination with the one or more of the information types i) to iii) being calculated in step c),

e) ranking the plurality of cancer types based on the classification score of step d), and f) predicting the specific type of cancer in said subject based on the ranking of step e).

Thus in one embodiment of the present invention, said method calculates said classification score based on a combination of the mutation status of step b) in combination with one or more of the information types i) to iii) of step c) such as for example by using the mutation status derived from step b) in combination with information types i) and/or ii) of step c), or by using a combination of the mutation status derived from step b) in combination with information type ii) of step c), or by using a combination of the mutation status derived from step b) in combination with information type iii) of step c), or by using combination of the mutation status derived from step b) in combination with information type iii) and one or more of the information types i) and ii) of step c), or by using a combination of the mutation status derived from step b) in combination with information types iii) and ii) of step c), or by using a combination of the mutation status derived from step b) in combination with information types iii) and i) of step c).

In a preferred embodiment of the invention, said classification score is calculated by using a combination of the mutation status of step b) in combination with one or more of information types ii) and iii) of step c).

In the method according to the present invention, said biological sequence may be a DNA, mRNA or protein sequence obtained from said bodily sample, wherein a DNA and/or mRNA sequence obtained from said bodily sample is more preferred.

In the methods of the present invention, both synonymous and non-synonymous mutations may be used for deriving said mutation status, wherein non-synonymous mutations are more preferred.

In one embodiment of the present invention, the mutation status of step b) is based on mutation status of genes that are recurrently mutated in association with cancer, such as for example the set of genes encoding the sequences of SEQ ID NO: 1 to 231, which is more preferred.

In one embodiment of he method according to the present invention, at least one of steps b) to c) is performed by a client, and wherein said client is capable of sending to a server one or more types of data used in the methods of the invention selected from the group consisting of genomic profile, mutation status, information type i), information type ii) information type iii). Typically, the server then returns to the client information about classification score and ranking.

In one embodiment of the method according to the present invention, at least one of steps b) to e) is performed by a server, and wherein said server is capable of receiving from a client one or more types of data according to claim 1 selected from the group consisting of genomic profile, mutation status, information type i), information type ii) information type iii). Typically, the server then returns to the client information about classification score and ranking.

In another aspect of the invention a computer program product is provided, said computer program product having instructions which when executed by a computing device or system causes the computing device or system to carry out the method according to the present invention.

In another aspect of the invention a data-processing system is provided having means for carrying out the method according to the present invention.

In another aspect of the invention a computer readable medium is provided having stored thereon a computer program product according to the present invention.

With the method according to the present invention it is possible to predict the tissue of origin (tissue type or cancer type) of a tumour metastasis with improved accuracy. This is particularly useful in the case of metastases of unknown origin or cancer of unknown primary. The improved accuracy provided by the present method can enable a more efficient clinical diagnosis of the primary tumour. Thereby the number of diagnostic tests needed to diagnose the primary cancer may be reduced, as well as the costs for the diagnostic procedure. Furthermore a more efficient diagnosis can aid to result in a faster and more efficient treatment which can reduce the mortality rate.

The methods of the current application may be used in connection with selecting a treatment regime by predicting the most likely origin of the cancer tissue and selecting a treatment regime based on this knowledge. As the predictive methods provide a rank with the most likely origins of the cancer, a treatment regime may be selected based on the top ranking cancer or on the topmost two or three cancers based on the classification score.

Another advantage of the method relates to the method providing a classification score ranking of each of the variety of cancer types predicted by the method. The ranking provides another aspect to the accuracy, since a user will know which types of cancer is ranking second and third on the ranked list, and will therefore have a good starting point for further clinical tests, in case the predicted cancer type with the highest rank cannot be diagnosed clinically in a patient.

Furthermore, said method can be based on a bodily sample obtained by using minimally invasive or non-invasive methods, such as for example a sample from a blood or urine sample. This will be beneficial for each individual patient as well as the efficiency of the medical services. It is further imagined that the invention will furthermore be applicable for screening and monitoring individuals with high risk of cancer, wherein cancer has not yet been diagnosed, due to the fact that it can be based on minimally invasive or non-invasive method.

In one embodiment, the method further includes calculation of a confidence score, for example a confidence score based on the difference between the top two ranking cancer types. This can help to indicate the if there are only minor differences in the classification score between the top ranking cancer types and thereby help to indicate if the second or third ranking cancer type should also be considered for clinical testing.

### DESCRIPTION OF DRAWINGS

The invention will in the following be described in greater detail with reference to the accompanying drawings:

FIG. 1. Outline of classifier development and validation of a method according to one embodiment of the invention. Development of final model, and feature selection, using five-fold internal cross validation, with final evaluation on a separate test set.

FIG. 2. Usage of a final method according to one embodiment of the invention on new samples. Somatic mutation data is used to infer the mutation status of a set of cancer genes and to calculate the distributions of either 6 or 96 classes of base substitutions (i.e. single base substitution wherein the two flanking bases are not taken into account, and single base substitution wherein the two flanking bases are taken into account). When CNV profiles are available, this is used to infer any copy number changes in the same set of cancer genes. These features are combined and provided to a set of random forest classifiers, one per cancer type, and the output classification scores are compared to establish the classification score rank of the possible cancer types for a given set of new samples.

FIG. 3. Performance of a method according to the invention in six and ten different tissues. Random forest ensembles were trained using the feature sets shown in the tables below each bar, and classification accuracy was evaluated by cross-validation. “Mutations” denote mutational status, “CNVs” denote copy number variation, “TripletBaseSubs” denote single base substitution wherein the two flanking bases are taken into account and “SingleBaseSubs” denote single base substitution wherein the two flanking bases are not taken into account. Adequate CNV data was available in only six of ten primary sites; thus we analyzed these six sites separately when considering CNV. Left: Classification accuracy when excluding CNV data and distinguishing between ten primary sites. Right: Classification accuracy when including CNV data and distinguishing between six primary sites. This figure shows that each of the three types of features enable better-than-random classification accuracy, and that combining these features enables even better accuracy. The overall accuracy for the combination of mutation status with either one of the types of single base substitution frequency was remarkably improved compared to methods using only one of these features, Trinucleotide base substitutions on their own have better accuracy than single base substitutions, and the best accuracy is achieved when these are combined with mutation status and CNVs. When CNVs are not available, the highest accuracy is attained with a combination of mutation status and trinucleotide base substitutions.

FIG. 4. Performance of final classifiers on the test data, according to the confidence score, defined as the difference between the top two ranking tissue classification scores. A. C: Classification accuracy increases with confidence score. Boxes and bars indicate the accuracy and 95% confidence interval for each bin of samples. Grey columns indicate the number of samples in each bin. B, D: Accuracy vs. fraction of samples called. Accuracy (solid line) and 95% confidence interval (grey region) of the corresponding fraction of tumours with highest confidence score. The fraction of tumours for which an accuracy of 95% can be achieved is shown by a grey box with whiskers at the bottom. These figures show that the confidence score can be used to select the most confident predictions; when CNVs are available (C, D), the top 75% of predictions with highest confidence have a combined accuracy of 95%. When CNVs are not available (A, B) our method has an accuracy of >85% on more than half the tumours.

FIG. 5. Performance of final classifiers in ranking primary sites. For each sample the primary sites were ordered from highest to lowest classification score, and each point indicates the percentage of samples (ordinate) where the correct primary cancer tissue type was found within the first specified number of tissue types (abscissa) when prioritising the tissue types by using our method (filled circles or line), by most frequent site (triangles) or at random (squares). The performance of the method on validation data sets with known distributions of primary cancer tissue types is estimated according to the “expected” site-specific accuracies based on the test data (crosses).

A: Performance of the model using mutation status in combination with trinucleotide single base substitution frequency (excluding CNV data) on the test set of ten primary cancer tissue types.

B: Performance of the method using mutation status in combination with trinucleotide single base substitution frequency and CNV data on the reduced test set of six primary cancer tissue types.

C: Performance of the method using mutation status in combination with trinucleotide single base substitution frequency (excluding CNV data) on the subset of COSMIC v70 that was not present in COSMIC v68 used for training.

D: Performance of the model using mutation status in combination with trinucleotide single base substitution frequency (excluding CNV data) on the SAFIR trial metastatic breast tumours.

A and B show that our methods correctly classify many more tumours than a random classifier would, or than a classifier that ranked the sites by their frequency in the data set would. C and D show that the accuracy on our validation data sets is comparable to the test set COSMIC 70. The actual validation accuracy is slightly lower than the “expected” accuracy calculated based on sensitivity of the specific cancer tissue type, this is not surprising due to differences in data generation and analysis, and the actual accuracy is still significantly better than a random classifier.

FIG. 6. Performance comparison of four machine learning methods used for the method of the present invention. The mean±SD of the area under the curve (AUC) of the receiver operating characteristics (ROC) curve is shown for each of ten primary tissue types, across 5-fold cross validation of stepwise logistic regression, support vector machines, artificial neural networks and random forests. This demonstrates that the method of the invention can be used successfully in combination with a range of different machine learning methods. The figure further demonstrates that in nine out of ten primary cancer tissue types, the random forest method achieved better classification performance than the other three methods, as evaluated by the AUC value, which is a well-established measure of binary classification performance.

FIG. 7. Confidence scores for all tumours in the cross-validation test sets, split into groups according to whether the top ranking primary cancer tissue type was correct or incorrect for the method using somatic mutation status in combination ii) single nucleotide substitutions taking the identity of the two flanking bases into account and further in combination with CNV information (type iii). The AUC value of the first method was 0.88. A similar plot was made for the method using somatic mutation status in combination with single nucleotide substitutions taking the identity of the two flanking bases. The AUC value of that method was 0.79. The AUC value reflects how well the confidence score can differentiate classifications that are likely to be correct from those that are less certain. The high AUC values show that our confidence score performs well in separating high confidence from less confident predictions.

FIG. 8. Flowchart for diagnosing cancer patients, including metastases of unknown origin (MUO) and cancer of unknown primary (CUP).

### DETAILED DESCRIPTION OF THE INVENTION

The term “cancer” as used herein is meant to encompass any cancer, neoplastic and preneoplastic disease.

By the term “classifier” as used herein is meant a method to identify to which set of categories (such as cancer types) a new observation belongs, where said method is based on a training set of data containing observations whose category membership is known. A classifier may be based on a machine learning method.

By the term “machine learning method” as used herein is meant an algorithm operated by a model based on inputs, and using that input to make predictions or decisions, rather than following explicitly programmed instructions. The machine (computer) is presented with example inputs and their desired outputs, and the goal is to learn a general rule that maps inputs to outputs. A commonly known example of such is email spam filtering, where the learning algorithm is presented with email messages labeled beforehand as “spam” or “not spam”, to produce a computer program that labels unseen messages as either spam or not.

“Biological sequences” according to the present invention may be any type of biological sequence of DNA, RNA or a protein sequence. Such sequences derived from cancer tissue may be compared to corresponding biological sequences from non-cancer tissue and used to derive or calculate different types of information, such as mutation status, single base substitution frequency, single base substitution frequency in triplets of nucleotide bases, and copy number variation (CNV).

By the term “bodily sample” as used herein is meant a sample of bodily material which includes biological sequences from the body wherefrom the sample is acquired. A bodily sample is for example a sample of bodily fluid or bodily tissue.

By the term “copy number variation (CNV)” as used herein is meant a change in the copy number status of a genomic region or segment such as a gene or set of adjacent genes, relative to a normal sample. Copy number variation results in the cell having an abnormal variation in the number of copies of one or more sections of the DNA.

By the term “normal sample” is meant a sample of bodily material of healthy or non-cancerous origin. Such a normal sample includes for example a sample of bodily tissue or bodily fluid. Common sources of normal samples are lymphocytes from peripheral blood samples, or tissue biopsies from healthy areas of the tissue or organ from where a matching tumour biopsy was taken. In the embodiments where the biological sequences are DNA sequences, a normal sample of non-cancerous origin may for example be derived from any non-cancerous bodily sample from a subject which comprises genomic DNA.

A normal sample may be a sample obtained from other human beings, in which case the biological sequences from a sample used for prediction of cancer tissue type in a subject are compared to a reference sequence from humans in general. The differences between the sequence from the subject and the sequence from the reference may reflect somatic mutations as well as population variations. Thus, in one embodiment of the present invention, one or more biological sequences of a sample to be tested in the method of the present invention are compared to biological sequences in a normal sample of another human being, such as for example one or more human reference sequences in databases.

When the normal sample is taken from the same subject, and even from the same tissue type as a tumour sample, the differences between the sequences from the tumour and normal sample reflect somatic mutations in the tumour genome compared to the germline genome of the subject. In a preferred embodiment of the present invention, the normal sample is derived from the same subject who is being tested using the method according to the invention. In another embodiment, the normal sample is derived from the same bodily tissue or fluid as the sample used for prediction of a specific type of cancer in a subject i.e. the comprising biological sequences from a tumour or cancer.

By the term “mutation status” as used herein is meant whether a gene is mutated or not compared to the gene in a normal sample. If any mutation is found, that gene will have status and be called as mutated. If no mutation is found, that gene will have status and be called as non-mutated.

By the term “single base substitution frequency” as used herein is meant the frequency of specific classes of single base substitutions or point mutations in an individual tumor genome. Single base mutation is a type of mutation that causes the replacement of a single base nucleotide with another nucleotide of the genetic material, DNA.

By the term “single base substitution frequency in triplets” as used herein is meant the frequency of specific classes of single base substitutions wherein the identity of the two flanking nucleotide bases are taken into account. One example is the frequency of substitution of the triplet of bases ACT wherein the middle base is substituted.

By the term “subject” is meant a subject which may potentially have cancer, such as for example an animal, a mammal, a primate or a human being.

### Prediction of Cancer Tissue Type

The present invention relates to a method wherein the mutational status in specific genes which are found to be mutated in cancer compared to the normal sample, is used in combination with one or more specific types of information selected from the group consisting of single base substitution frequency wherein the two flanking bases are not taking into account (information type i), single base substitution frequency in triplets of nucleotide bases wherein the two flanking bases are taking into account (information type ii); and copy number variation in genomic regions and/or genes and/or sets of genes compared to the copy number of the same genomic regions and/or genes and/or sets of genes in a normal sample, such as a healthy sample or a non-cancerous sample (information type iii). The mutation status and the remaining types of information as mentioned above can be calculated based on data of biological sequences from a cancerous cell. The inventors have surprisingly found that the combination of these specific types of information derived from cancer result in a high overall performance in a method for prediction of a specific cancer type in a subject, wherein the method provides a ranking of different cancer types.

In one embodiment of the present invention, the method calculates said classification score using at least a combination of mutation status with calculated single base substitution frequency information types i) and/or ii) of step c), for example the method may only use a combination of mutation status with single base substitution frequency information type as i) and/or ii) of step c).

In another embodiment of the present invention, the method calculates said classification score using at least a combination of mutation status with calculated single base substitution frequency information type ii) of step c), for example the method may only use only a combination of mutation status with calculated single base substitution frequency information type ii) of step c).

The inventors of the present method have surprisingly found that the predictive accuracy of the method can be increased by calculating copy number variation of genomic regions, and using this information in a method for predicting cancer according to the present invention.

In one embodiment of the present invention, the method calculates the classification score for each cancer type based on a combination of the mutation status derived from step b) in combination with information type iii) and one or more of the information types i) and ii) of step c), for example the method can in one embodiment calculate a classification score for each cancer type based on a combination of the mutation status derived from step b) in combination with information types iii) and i) of step c).

In an even more preferred embodiment, the method according to the invention calculates the classification score for each cancer type based on a combination of the mutation status derived from step b) in combination with information types iii) and ii) of step c).

A number of different biological sequences exist which may be used to derive information that is useful as input for the method according to the present invention. In one embodiment of the present invention, a biological sequence is a DNA, RNA or protein sequence obtained from a bodily sample comprising cancerous or non-cancerous material. In one other embodiment of the present invention, the biological sequence is a DNA and/or a mRNA sequence.

In a more preferred embodiment of the present invention, the biological sequence is a DNA sequence, such as a genomic sequence, a gene sequence, an exome sequence or a cDNA sequence.

The information derived from biological sequences and used for calculation of classification score according to the present invention relies on a comparison to biological sequences in a normal sample which comprises healthy or non-cancerous material, such as bodily fluid or tissue. However, natural variation may occur in corresponding biological sequences between subjects of given population. If the normal sample is derived from the same subject who is to be tested using the method of the present invention, it is envisaged that the predictive performance will increase due to a more efficient comparison of biological sequences. Therefore, in one embodiment of the present invention, biological sequences for both a normal sample and a bodily sample comprising cancer tissue or cancer biological sequences are provided from the same subject and used for the calculation of information types i) to iii) of step c) and for the mutation status of step b).

The predictive accuracy can be further improved by using additional data derived from cancerous samples. In one embodiment of the present invention, the method further includes the use of information of additional clinical or pathological features such as expression of tissue-characteristic mRNA or proteins, or the location of the metastasis in the body of a subject.

### Derivation of Mutation Status for Use in the Method of the Invention

Mutational status of genes which are mutated in cancer can provide valuable information regarding the type of cancer found in cancer cells or biological sequences of a bodily sample.

Mutations in a biological sequence can be determined by using methods which are commonly known in the art, such as DNA, RNA or protein sequencing, such as for example whole genome sequencing, targeted sequencing, exomic sequencing or hybridization-based methods and comparing the data to a normal sample.

Typically, a first step of identifying mutations in a biological sample involves sequence alignment of biological sequences derived from a sample of cancer material with a corresponding biological sequence from a reference or normal (or non-tumour) sample using conventional methods known in the art, such as, BLAST (Altschul et al 1990), BWA (Li and Durbin, 2009), Bowtie (Langmead et al 2009), or various combinations of substring index searching and/or dynamic programming. Based on the alignment, differences in the biological sequences can be used to determine if there are specific mutations or substitutions, insertions, deletions, or changed locations in the biological sequence of a cancer cell compared to the normal sample. Thus in a preferred embodiment of the present invention, the mutation status of biological sequences from a sample is derived by alignment of biological sequences.

In one embodiment of the present invention, the mutation status of a gene mutated in cancer is derived from a comparison of mRNA or cDNA sequences of the tumour specimen and/or normal specimen, and by mapping these changes to specific genes or genomic positions.

In another embodiment of the present invention, the mutation status of a gene mutated in cancer is derived by comparison of protein sequences by identifying amino acid positions in which a specific amino acid or sequence of amino acids has been modified, inserted, deleted, or changed location in the cancer specimen relative to the normal specimen, and by mapping these changes to specific genes or genomic positions.

In a preferred embodiment of the present invention, mutation status of a gene mutated in cancer is derived by comparison of genomic DNA sequences derived from a cancerous specimen and from a corresponding normal (or non-cancer) specimen, and by mapping/aligning these sequences to specific genomic locations.

When the biological sequences are DNA sequences, mutational status may be identified by obtaining genomic DNA sequences derived from a tumour specimen and from a corresponding normal (or non-tumour) specimen, and by mapping/aligning these sequences to specific genomic locations using programs such as bowtie (Langmead et al 2012), and by identifying genomic positions in which a specific base or sequence of bases has been modified, inserted, deleted, or changed location in the tumour specimen relative to the normal specimen, using programs such as MuTect (Cibulskis et al 2013) or VarScan (Koboldt et al 2012).

The mutation status of any gene can be evaluated and used in the method according to the present invention. However, mutations may occur which are not related to cancer. Such mutations may result in a reduced accuracy of prediction. Therefore, the method of present invention preferably uses mutation status of genes that are mutated in association with cancer. One way of determining such genes is by using the method or components of the methods as described by Lawrence et al 2013, which involves identifying genes that are mutated more frequently than expected from the background mutation rate in any individual cancer type, as identified through genomic analysis of several individual tumours. In one embodiment of the present invention, the mutation status is derived in genes that are mutated in cancer, wherein the selected genes are mutated more frequently than expected from the background mutation rate in any individual cancer type, as identified through genomic analysis of several individual tumours.

In an even more preferred embodiment, the method according to the present invention uses mutation status of a specific set of selected genes which have been determined as being frequently mutated in cancer such as the set of genes encoding the coding DNA sequences (CDS) of SEQ ID NO: 1 to 231 or in the sequences of SEQ ID NO: 1 to 231, which are shown in Table 1 below. The column named HGNC ID(s) below refers to human genome version GRCh38.

TABLE 1

| | Ensembl | HGNC | Entrez |
|---|---|---|---|
| SEQ ID NO | Gene ID | ID(s) | Gene ID |
| SEQ ID NO: 1 | ENSG00000122729 | HGNC: 117 | 48 |
| SEQ ID NO: 2 | ENSG00000135503 | HGNC: 172 | 91 |
| SEQ ID NO: 3 | ENSG00000101126 | HGNC: 15766 | 23394 |
| SEQ ID NO: 4 | ENSG00000129474 | HGNC: 20250 | 84962 |
| SEQ ID NO: 5 | ENSG00000142208 | HGNC: 391 | 207 |
| SEQ ID NO: 6 | ENSG00000171094 | HGNC: 427 | 238 |
| SEQ ID NO: 7 | ENSG00000239382 | HGNC: 28243 | 84964 |
| SEQ ID NO: 8 | ENSG00000198796 | HGNC: 20565 | 115701 |
| SEQ ID NO: 9 | ENSG00000151150 | HGNC: 494 | 288 |
| SEQ ID NO: 10 | ENSG00000134982 | HGNC: 583 | 324 |
| SEQ ID NO: 11 | ENSG00000160007 | HGNC: 4591 | 2909 |
| SEQ ID NO: 12 | ENSG00000117713 | HGNC: 11110 | 8289 |
| SEQ ID NO: 13 | ENSG00000189079 | HGNC: 18037 | 196528 |
| SEQ ID NO: 14 | ENSG00000150347 | HGNC: 17362 | 84159 |
| SEQ ID NO: 15 | ENSG00000171456 | HGNC: 18318 | 171023 |
| SEQ ID NO: 16 | ENSG00000149311 | HGNC: 795 | 472 |
| SEQ ID NO: 17 | ENSG00000168646 | HGNC: 904 | 8313 |
| SEQ ID NO: 18 | ENSG00000160862 | HGNC: 910 | 563 |
| SEQ ID NO: 19 | ENSG00000166710 | HGNC: 914 | 567 |
| SEQ ID NO: 20 | ENSG00000163930 | HGNC: 950 | 8314 |
| SEQ ID NO: 21 | ENSG00000029363 | HGNC: 16863 | 9774 |
| SEQ ID NO: 22 | ENSG00000183337 | HGNC: 20893 | 54880 |
| SEQ ID NO: 23 | ENSG00000157764 | HGNC: 1097 | 673 |
| SEQ ID NO: 24 | ENSG00000012048 | HGNC: 1100 | 672 |
| SEQ ID NO: 25 | ENSG00000187068 | HGNC: 33731 | 285382 |
| SEQ ID NO: 26 | ENSG00000112186 | HGNC: 20039 | 10486 |
| SEQ ID NO: 27 | ENSG00000198286 | HGNC: 16393 | 84433 |
| SEQ ID NO: 28 | ENSG00000064012 | HGNC: 1509 | 841 |
| SEQ ID NO: 29 | ENSG00000067955 | HGNC: 1539 | 865 |
| SEQ ID NO: 30 | ENSG00000147144 | HGNC: 28910 | 90060 |
| SEQ ID NO: 31 | ENSG00000108091 | HGNC: 18782 | 8030 |
| SEQ ID NO: 32 | ENSG00000110092 | HGNC: 1582 | 595 |
| SEQ ID NO: 33 | ENSG00000158473 | HGNC: 1637 | 912 |
| SEQ ID NO: 34 | ENSG00000125726 | HGNC: 11937 | 970 |
| SEQ ID NO: 35 | ENSG00000007312 | HGNC: 1699 | 974 |
| SEQ ID NO: 36 | ENSG00000004897 | HGNC: 1728 | 996 |
| SEQ ID NO: 37 | ENSG00000039068 | HGNC: 1748 | 999 |
| SEQ ID NO: 38 | ENSG00000167258 | HGNC: 24224 | 51755 |
| SEQ ID NO: 39 | ENSG00000135446 | HGNC: 1773 | 1019 |
| SEQ ID NO: 40 | ENSG00000124762 | HGNC: 1784 | 1026 |
| SEQ ID NO: 41 | ENSG00000111276 | HGNC: 1785 | 1027 |
| SEQ ID NO: 42 | ENSG00000147889 | HGNC: 1787 | 1029 |
| SEQ ID NO: 43 | ENSG00000245848 | HGNC: 1833 | 1050 |
| SEQ ID NO: 44 | ENSG00000111642 | HGNC: 1919 | 1108 |
| SEQ ID NO: 45 | ENSG00000100888 | HGNC: 20153 | 57680 |
| SEQ ID NO: 46 | ENSG00000176571 | HGNC: 26663 | 168975 |
| SEQ ID NO: 47 | ENSG00000130635 | HGNC: 2209 | 1289 |
| SEQ ID NO: 48 | ENSG00000080573 | HGNC: 14864 | 50509 |
| SEQ ID NO: 49 | ENSG00000005339 | HGNC: 2348 | 1387 |
| SEQ ID NO: 50 | ENSG00000102974 | HGNC: 13723 | 10664 |
| SEQ ID NO: 51 | ENSG00000168036 | HGNC: 2514 | 1499 |
| SEQ ID NO: 52 | ENSG00000158290 | HGNC: 2555 | 8450 |
| SEQ ID NO: 53 | ENSG00000257923 | HGNC: 2557 | 1523 |
| SEQ ID NO: 54 | ENSG00000215301 | HGNC: 2745 | 1654 |
| SEQ ID NO: 55 | ENSG00000108654 | HGNC: 2746 | 1655 |
| SEQ ID NO: 56 | ENSG00000131504 | HGNC: 2876 | 1729 |
| SEQ ID NO: 57 | ENSG00000083520 | HGNC: 20604 | 22894 |
| SEQ ID NO: 58 | ENSG00000187957 | HGNC: 24456 | 92737 |
| SEQ ID NO: 59 | ENSG00000119772 | HGNC: 2978 | 1788 |
| SEQ ID NO: 60 | ENSG00000146648 | HGNC: 3236 | 1956 |
| SEQ ID NO: 61 | ENSG00000125977 | HGNC: 3266 | 8894 |
| SEQ ID NO: 62 | ENSG00000163435 | HGNC: 3318 | 1999 |
| SEQ ID NO: 63 | ENSG00000100393 | HGNC: 3373 | 2033 |
| SEQ ID NO: 64 | ENSG00000142627 | HGNC: 3386 | 1969 |
| SEQ ID NO: 65 | ENSG00000141736 | HGNC: 3430 | 2064 |
| SEQ ID NO: 66 | ENSG00000065361 | HGNC: 3431 | 2065 |
| SEQ ID NO: 67 | ENSG00000104884 | HGNC: 3434 | 2068 |
| SEQ ID NO: 68 | ENSG00000106462 | HGNC: 3527 | 2146 |
| SEQ ID NO: 69 | ENSG00000092820 | HGNC: 12691 | 7430 |
| SEQ ID NO: 70 | ENSG00000183508 | HGNC: 24712 | 54855 |
| SEQ ID NO: 71 | ENSG00000083857 | HGNC: 3595 | 2195 |
| SEQ ID NO: 72 | ENSG00000109670 | HGNC: 16712 | 55294 |
| SEQ ID NO: 73 | ENSG00000066468 | HGNC: 3689 | 2263 |
| SEQ ID NO: 74 | ENSG00000068078 | HGNC: 3690 | 2261 |
| SEQ ID NO: 75 | ENSG00000143631 | HGNC: 3748 | 2312 |
| SEQ ID NO: 76 | ENSG00000122025 | HGNC: 3765 | 2322 |
| SEQ ID NO: 77 | ENSG00000129514 | HGNC: 5021 | 3169 |
| SEQ ID NO: 78 | ENSG00000164379 | HGNC: 20951 | 94234 |
| SEQ ID NO: 79 | ENSG00000165694 | HGNC: 8079 | 90167 |
| SEQ ID NO: 80 | ENSG00000107485 | HGNC: 4172 | 2625 |
| SEQ ID NO: 81 | ENSG00000120063 | HGNC: 4381 | 10672 |
| SEQ ID NO: 82 | ENSG00000111670 | HGNC: 29670 | 79158 |
| SEQ ID NO: 83 | ENSG00000120053 | HGNC: 4432 | 2805 |
| SEQ ID NO: 84 | ENSG00000169919 | HGNC: 4696 | 2990 |
| SEQ ID NO: 85 | ENSG00000168298 | HGNC: 4718 | 3008 |
| SEQ ID NO: 86 | ENSG00000206503 | HGNC: 4931 | 3105 |
| SEQ ID NO: 87 | ENSG00000234745 | HGNC: 4932 | 3106 |
| SEQ ID NO: 88 | ENSG00000174775 | HGNC: 5173 | 3265 |
| SEQ ID NO: 89 | ENSG00000138413 | HGNC: 5382 | 3417 |
| SEQ ID NO: 90 | ENSG00000182054 | HGNC: 5383 | 3418 |
| SEQ ID NO: 91 | ENSG00000168685 | HGNC: 6024 | 3575 |
| SEQ ID NO: 92 | ENSG00000153487 | HGNC: 6062 | 3621 |
| SEQ ID NO: 93 | ENSG00000165458 | HGNC: 6080 | 3636 |
| SEQ ID NO: 94 | ENSG00000138785 | HGNC: 25067 | 57117 |
| SEQ ID NO: 95 | ENSG00000205339 | HGNC: 9852 | 10527 |
| SEQ ID NO: 96 | ENSG00000137265 | HGNC: 6119 | 3662 |
| SEQ ID NO: 97 | ENSG00000143772 | HGNC: 6179 | 3707 |
| SEQ ID NO: 98 | ENSG00000162434 | HGNC: 6190 | 3716 |
| SEQ ID NO: 99 | ENSG00000126012 | HGNC: 11114 | 8242 |
| SEQ ID NO: 100 | ENSG00000147050 | HGNC: 12637 | 7403 |
| SEQ ID NO: 101 | ENSG00000079999 | HGNC: 23177 | 9817 |
| SEQ ID NO: 102 | ENSG00000197993 | HGNC: 6308 | 3792 |
| SEQ ID NO: 103 | ENSG00000157404 | HGNC: 6342 | 3815 |
| SEQ ID NO: 104 | ENSG00000145332 | HGNC: 18644 | 57563 |
| SEQ ID NO: 105 | ENSG00000118058 | HGNC: 7132 | 4297 |
| SEQ ID NO: 106 | ENSG00000272333 | HGNC: 15840 | 9757 |
| SEQ ID NO: 107 | ENSG00000055609 | HGNC: 13726 | 58508 |
| SEQ ID NO: 108 | ENSG00000167548 | HGNC: 7133 | 8085 |
| SEQ ID NO: 109 | ENSG00000133703 | HGNC: 6407 | 3845 |
| SEQ ID NO: 110 | ENSG00000188501 | HGNC: 15583 | 197021 |
| SEQ ID NO: 111 | ENSG00000169032 | HGNC: 6840 | 5604 |
| SEQ ID NO: 112 | ENSG00000065559 | HGNC: 6844 | 6416 |
| SEQ ID NO: 113 | ENSG00000095015 | HGNC: 6848 | 4214 |
| SEQ ID NO: 114 | ENSG00000011566 | HGNC: 6865 | 8491 |
| SEQ ID NO: 115 | ENSG00000184634 | HGNC: 11957 | 9968 |
| SEQ ID NO: 116 | ENSG00000112282 | HGNC: 2372 | 9439 |
| SEQ ID NO: 117 | ENSG00000105976 | HGNC: 7029 | 4233 |
| SEQ ID NO: 118 | ENSG00000174197 | HGNC: 14010 | 23269 |
| SEQ ID NO: 119 | ENSG00000133808 | HGNC: 25933 | 84953 |
| SEQ ID NO: 120 | ENSG00000133131 | HGNC: 23485 | 79710 |
| SEQ ID NO: 121 | ENSG00000005381 | HGNC: 7218 | 4353 |
| SEQ ID NO: 122 | ENSG00000198793 | HGNC: 3942 | 2475 |
| SEQ ID NO: 123 | ENSG00000169876 | HGNC: 16800 | 140453 |
| SEQ ID NO: 124 | ENSG00000101825 | HGNC: 7539 | 25878 |
| SEQ ID NO: 125 | ENSG00000118513 | HGNC: 7545 | 4602 |
| SEQ ID NO: 126 | ENSG00000134323 | HGNC: 7559 | 4613 |
| SEQ ID NO: 127 | ENSG00000172936 | HGNC: 7562 | 4615 |
| SEQ ID NO: 128 | ENSG00000141052 | HGNC: 16067 | 93649 |
| SEQ ID NO: 129 | ENSG00000141027 | HGNC: 7672 | 9611 |
| SEQ ID NO: 130 | ENSG00000196712 | HGNC: 7765 | 4763 |
| SEQ ID NO: 131 | ENSG00000116044 | HGNC: 7782 | 4780 |
| SEQ ID NO: 132 | ENSG00000148400 | HGNC: 7881 | 4851 |
| SEQ ID NO: 133 | ENSG00000181163 | HGNC: 7910 | 4869 |
| SEQ ID NO: 134 | ENSG00000213281 | HGNC: 7989 | 4893 |
| SEQ ID NO: 135 | ENSG00000165671 | HGNC: 14234 | 64324 |
| SEQ ID NO: 136 | ENSG00000074527 | HGNC: 13658 | 59277 |
| SEQ ID NO: 137 | ENSG00000143552 | HGNC: 29915 | 91181 |
| SEQ ID NO: 138 | ENSG00000181961 | HGNC: 15153 | 81327 |
| SEQ ID NO: 139 | ENSG00000181001 | HGNC: 14853 | 79473 |
| SEQ ID NO: 140 | ENSG00000169918 | HGNC: 20718 | 161725 |
| SEQ ID NO: 141 | ENSG00000121274 | HGNC: 30758 | 64282 |
| SEQ ID NO: 142 | ENSG00000163939 | HGNC: 30064 | 55193 |
| SEQ ID NO: 143 | ENSG00000169564 | HGNC: 8647 | 5093 |
| SEQ ID NO: 144 | ENSG00000164494 | HGNC: 23041 | 57107 |
| SEQ ID NO: 145 | ENSG00000156531 | HGNC: 18145 | 84295 |
| SEQ ID NO: 146 | ENSG00000121879 | HGNC: 8975 | 5290 |
| SEQ ID NO: 147 | ENSG00000145675 | HGNC: 8979 | 5295 |
| SEQ ID NO: 148 | ENSG00000177084 | HGNC: 9177 | 5426 |
| SEQ ID NO: 149 | ENSG00000110777 | HGNC: 9211 | 5450 |
| SEQ ID NO: 150 | ENSG00000028277 | HGNC: 9213 | 5452 |
| SEQ ID NO: 151 | ENSG00000170836 | HGNC: 9277 | 8493 |
| SEQ ID NO: 152 | ENSG00000105568 | HGNC: 9302 | 5518 |
| SEQ ID NO: 153 | ENSG00000119414 | HGNC: 9323 | 5537 |
| SEQ ID NO: 154 | ENSG00000057657 | HGNC: 9346 | 639 |
| SEQ ID NO: 155 | ENSG00000171862 | HGNC: 9588 | 5728 |
| SEQ ID NO: 156 | ENSG00000179295 | HGNC: 9644 | 5781 |
| SEQ ID NO: 157 | ENSG00000112531 | HGNC: 21100 | 9444 |
| SEQ ID NO: 158 | ENSG00000172476 | HGNC: 18283 | 142684 |
| SEQ ID NO: 159 | ENSG00000136238 | HGNC: 9801 | 5879 |
| SEQ ID NO: 160 | ENSG00000164754 | HGNC: 9811 | 5885 |
| SEQ ID NO: 161 | ENSG00000145715 | HGNC: 9871 | 5921 |
| SEQ ID NO: 162 | ENSG00000139687 | HGNC: 9884 | 5925 |
| SEQ ID NO: 163 | ENSG00000182872 | HGNC: 9896 | 8241 |
| SEQ ID NO: 164 | ENSG00000106615 | HGNC: 10011 | 6009 |
| SEQ ID NO: 165 | ENSG00000067560 | HGNC: 667 | 387 |
| SEQ ID NO: 166 | ENSG00000143622 | HGNC: 10023 | 6016 |
| SEQ ID NO: 167 | ENSG00000122406 | HGNC: 10360 | 6125 |
| SEQ ID NO: 168 | ENSG00000115268 | HGNC: 10388 | 6209 |
| SEQ ID NO: 169 | ENSG00000140988 | HGNC: 10404 | 6187 |
| SEQ ID NO: 170 | ENSG00000187257 | HGNC: 24765 | 222194 |
| SEQ ID NO: 171 | ENSG00000159216 | HGNC: 10471 | 861 |
| SEQ ID NO: 172 | ENSG00000186350 | HGNC: 10477 | 6256 |
| SEQ ID NO: 173 | ENSG00000151835 | HGNC: 10519 | 26278 |
| SEQ ID NO: 174 | ENSG00000174175 | HGNC: 10721 | 6403 |
| SEQ ID NO: 175 | ENSG00000197641 | HGNC: 8944 | 5275 |
| SEQ ID NO: 176 | ENSG00000181555 | HGNC: 18420 | 29072 |
| SEQ ID NO: 177 | ENSG00000143379 | HGNC: 10761 | 9869 |
| SEQ ID NO: 178 | ENSG00000115524 | HGNC: 10768 | 23451 |
| SEQ ID NO: 179 | ENSG00000118515 | HGNC: 10810 | 6446 |
| SEQ ID NO: 180 | ENSG00000089163 | HGNC: 14932 | 23409 |
| SEQ ID NO: 181 | ENSG00000079215 | HGNC: 10941 | 6507 |
| SEQ ID NO: 182 | ENSG00000091138 | HGNC: 3018 | 1811 |
| SEQ ID NO: 183 | ENSG00000143036 | HGNC: 28689 | 126969 |
| SEQ ID NO: 184 | ENSG00000188687 | HGNC: 18168 | 57835 |
| SEQ ID NO: 185 | ENSG00000175387 | HGNC: 6768 | 4087 |
| SEQ ID NO: 186 | ENSG00000141646 | HGNC: 6770 | 4089 |
| SEQ ID NO: 187 | ENSG00000127616 | HGNC: 11100 | 6597 |
| SEQ ID NO: 188 | ENSG00000099956 | HGNC: 11103 | 6598 |
| SEQ ID NO: 189 | ENSG00000072501 | HGNC: 11111 | 8243 |
| SEQ ID NO: 190 | ENSG00000108055 | HGNC: 2468 | 9126 |
| SEQ ID NO: 191 | ENSG00000109762 | HGNC: 21883 | 83891 |
| SEQ ID NO: 192 | ENSG00000115904 | HGNC: 11187 | 6654 |
| SEQ ID NO: 193 | ENSG00000164736 | HGNC: 18122 | 64321 |
| SEQ ID NO: 194 | ENSG00000065526 | HGNC: 17575 | 23013 |
| SEQ ID NO: 195 | ENSG00000121067 | HGNC: 11254 | 8405 |
| SEQ ID NO: 196 | ENSG00000161547 | HGNC: 10783 | 6427 |
| SEQ ID NO: 197 | ENSG00000101972 | HGNC: 11355 | 10735 |
| SEQ ID NO: 198 | ENSG00000118046 | HGNC: 11389 | 6794 |
| SEQ ID NO: 199 | ENSG00000204344 | HGNC: 11398 | 8859 |
| SEQ ID NO: 200 | ENSG00000111450 | HGNC: 3403 | 2054 |
| SEQ ID NO: 201 | ENSG00000168394 | HGNC: 43 | 6890 |
| SEQ ID NO: 202 | ENSG00000108239 | HGNC: 29082 | 23232 |
| SEQ ID NO: 203 | ENSG00000177565 | HGNC: 29529 | 79718 |
| SEQ ID NO: 204 | ENSG00000135111 | HGNC: 11602 | 6926 |
| SEQ ID NO: 205 | ENSG00000154582 | HGNC: 11617 | 6921 |
| SEQ ID NO: 206 | ENSG00000148737 | HGNC: 11641 | 6934 |
| SEQ ID NO: 207 | ENSG00000166046 | HGNC: 28627 | 255394 |
| SEQ ID NO: 208 | ENSG00000163239 | HGNC: 25316 | 126668 |
| SEQ ID NO: 209 | ENSG00000168769 | HGNC: 25941 | 54790 |
| SEQ ID NO: 210 | ENSG00000163513 | HGNC: 11773 | 7048 |
| SEQ ID NO: 211 | ENSG00000232810 | HGNC: 11892 | 7124 |
| SEQ ID NO: 212 | ENSG00000157873 | HGNC: 11912 | 8764 |
| SEQ ID NO: 213 | ENSG00000141510 | HGNC: 11998 | 7157 |
| SEQ ID NO: 214 | ENSG00000067369 | HGNC: 11999 | 7158 |
| SEQ ID NO: 215 | ENSG00000088325 | HGNC: 1249 | 22974 |
| SEQ ID NO: 216 | ENSG00000131323 | HGNC: 12033 | 7187 |
| SEQ ID NO: 217 | ENSG00000113595 | HGNC: 660 | 373 |
| SEQ ID NO: 218 | ENSG00000165699 | HGNC: 12362 | 7248 |
| SEQ ID NO: 219 | ENSG00000131044 | HGNC: 16118 | 164395 |
| SEQ ID NO: 220 | ENSG00000204193 | HGNC: 31454 | 255220 |
| SEQ ID NO: 221 | ENSG00000160201 | HGNC: 12453 | 7307 |
| SEQ ID NO: 222 | ENSG00000134086 | HGNC: 12687 | 7428 |
| SEQ ID NO: 223 | ENSG00000132970 | HGNC: 12734 | 10810 |
| SEQ ID NO: 224 | ENSG00000184937 | HGNC: 12796 | 7490 |
| SEQ ID NO: 225 | ENSG00000163092 | HGNC: 14303 | 129446 |
| SEQ ID NO: 226 | ENSG00000082898 | HGNC: 12825 | 7514 |
| SEQ ID NO: 227 | ENSG00000140836 | HGNC: 777 | 463 |
| SEQ ID NO: 228 | ENSG00000196263 | HGNC: 23226 | 57573 |
| SEQ ID NO: 229 | ENSG00000177842 | HGNC: 28742 | 253639 |
| SEQ ID NO: 230 | ENSG00000141579 | HGNC: 25843 | 79755 |
| SEQ ID NO: 231 | ENSG00000121988 | HGNC: 25249 | 84083 |

A mutation or substitution of a nucleotide in a biological sequence is denoted “non-synonymous” or “non-silent” when the mutation or substitution results in a change of an amino acid in, or disruption of, the corresponding protein sequence which may be formed by the expression of a mutated gene. Correspondingly, when the mutation or substitution does not result in a change of an amino acid in the corresponding protein sequence, the mutation or substitution is denoted “synonymous”. In one embodiment of the present invention, both synonymous and non-synonymous mutations are used in the derivation of mutation status in a gene. In such embodiments a gene is indicated as mutated if one or more synonymous or non-synonymous mutations are determined in the gene compared to the gene of a normal sample.

Non-synonymous mutations may lead to functional or structural changes in the corresponding protein which in turn may alter the function of a cell such as seen in relation to cancer. Synonymous mutations may not give rise to any changes of properties of a corresponding protein. In a more preferred embodiment of the present invention, only non-synonymous mutations are used in the derivation of mutation status in a gene. In such embodiments a gene is only indicated as mutated if one or more non-synonymous mutations are determined in the exons of a gene compared to the gene of a normal sample.

In a more preferred embodiment, only non-synonymous mutations in exons of the genes, such as in the coding DNA sequences of SEQ ID NO:1 to 231 of Table 1 are used in the derivation of mutation status for use in the prediction method of the present invention.

### Information On Single Base Substitution for Use in the Method of the Invention

Single base substitutions are often associated with cancer. DNA comprises the nucleotide bases cytosine (C), guanine (G), thymine (T) and adenine (A). There are thus 12 possible different single base substitution classes when the identities of the flanking bases are not taken into account or used for classification. These 12 different classes may be selected from the group consisting of C to G, C to A, C to T, T to A, T to C, T to G, G to A, G to C, G to T, A to C, A to G and A to T.

Each type of the different single base substitution classes may be associated with different types of cancer. In one embodiment of the present invention, the calculation of information types i) of step c) performed by using the frequency of observations of one or more of the 12 base substitution classes as defined above in the biological sequence of a bodily sample. In a specific embodiment of the present invention, the calculation of information types i) of step c) involves the calculation of the relative contribution of one or more of 12 base substitution classes as defined above in biological sequences of a bodily sample.

Single base substitutions in a biological sequence can be determined by using DNA or RNA sequencing methods, wherein DNA sequencing is more preferred.

For a given tumour sample, the single base substitution frequency for a given class of base substitution can be calculated by counting the number of genomic locations in which that class of base substitution is identified. In another embodiment, this resulting number is divided by the total number of identified single base substitutions of any class.

Since DNA is commonly found as a base-paired double strand of nucleotides, a substitution in one strand is usually found in combination with a substitution in the corresponding complementary strand. Therefore, in some embodiments of the present invention, information of single base substitution frequency may be calculated by counting only the pyrimidine of the germline Watson-Crick base pair, and thus reducing the number of classes to six different classes of single base substitutions selected from the group consisting of C to A, C to G, C to T, T to A, T to C and T to G. In one embodiment of the present invention, the calculation of information types i) of step c) involves the calculation of the relative contribution of one or more of the 6 base substitution classes as defined above using the pyrimidine of the germline Watson-Crick base pair.

Single base substitution frequency may alternatively be calculated by using only the purine of the germline Watson-Crick base pair. Therefore, in some embodiments of the present invention, information of single base substitution frequency may be calculated by using only the purine of the germline Watson-Crick base pair, and thus reducing the number of classes to six different classes of single base substitutions selected from the group consisting of A to C, A to T, A to G, G to T, G to C and G to A. In one embodiment of the present invention, the calculation of information types i) of step c) involves the calculation of the relative contribution of one or more of the 6 base substitution classes as defined above using the purine of the germline Watson-Crick base pair, such as for example all 6 base substitution classes.

Single base substitution frequency in specific triplets of nucleotide bases, wherein the identity of the two bases flanking the substituted base is taken into account has further been associated with cancer. When the identity of the two flanking bases is taken into account, there are 192 different classes of single base substitutions which are possible.

In another embodiment of the present invention, the single base substitution frequency in specific triplets of nucleotide bases (information type ii) of step c) is calculated by using only the purine of the germline Watson-Crick base pair for the middle single base substitution, and thus reducing the number of classes to 96 different classes of single base substitutions. Following this scheme, the middle substituted base can vary between the 6 types of substitutions selected from the group consisting of A to C, A to T, A to G, G to T, G to C and G to A, and the identity of each of the two flanking bases may be selected from A, C, G or T.

In another embodiment of the present invention, the single base substitution frequency in specific triplets of nucleotide bases (information type ii) of step c) is calculated by using only the pyrimidine of the germline Watson-Crick base pair for the middle single base substitution, and thus reducing the number of classes to 96 different classes of single base substitutions. Following this scheme, the middle substituted base in DNA can vary between the 6 types of substitutions selected from the group consisting of C to A, C to G, C to T, T to A, T to C and T to G and the identity of each of the two flanking bases may be selected from A, C, G or T. Thus in one embodiment of the present invention, the single base substitution frequency in specific triplets of nucleotide bases (information types i) of step c) is calculated by using only the pyrimidine of the germline Watson-Crick base pair for the single base substitution, and using one or more of the resulting 96 different classes of single base substitutions selected from the group consisting of the triplets of nucleotides of Table 2 below.

TABLE 2
96 different trinucleotide classes obtained by
using only the pyrimidine of the germline
Watson-Crick base pair for the middle single
base substitution.

| | | | |
|---|---|---|---|
| ACA to AAA | CCC to CAC | GCG to GAG | TCT to TAT |
| CCA to CAA | GCC to GAC | TCG to TAG | ATT to AAT |
| GCA to GAA | TCC to TAC | ATG to AAG | CTT to CAT |
| TCA to TAA | ATC to AAC | CTG to CAG | GTT to GAT |
| ATA to AAA | CTC to CAC | GTG to GAG | TTT to TAT |
| CTA to CAA | GTC to GAC | TTG to TAG | ATT to ACT |
| GTA to GAA | TTC to TAC | ATG to ACG | CTT to CCT |
| TTA to TAA | ATC to ACC | CTG to CCG | GTT to GCT |
| ATA to ACA | CTC to CCC | GTG to GCG | TTT to TCT |
| CTA to CCA | GTC to GCC | TTG to TCG | ACT to AGT |
| GTA to GCA | TTC to TCC | ACG to AGG | CCT to CGT |
| TTA to TCA | ACC to AGC | CCG to CGG | GCT to GGT |
| ACA to AGA | CCC to CGC | GCG to GGG | TCT to TGT |
| CCA to CGA | GCC to GGC | TCG to TGG | ATT to AGT |
| GCA to GGA | TCC to TGC | ATG to AGG | CTT to CGT |
| TCA to TGA | ATC to AGC | CTG to CGG | GTT to GGT |
| ATA to AGA | CTC to CGC | GTG to GGG | TTT to TGT |
| CTA to CGA | GTC to GGC | TTG to TGG | ACT to ATT |
| GTA to GGA | TTC to TGC | ACG to ATG | CCT to CTT |
| TTA to TGA | ACC to ATC | CCG to CTG | GCT to GTT |
| ACA to ATA | CCC to CTC | GCG to GTG | TCT to TTT |
| CCA to CTA | GCC to GTC | TCG to TTG | |
| GCA to GTA | TCC to TTC | ACT to AAT | |
| TCA to TTA | ACG to AAG | CCT to CAT | |
| ACC to AAC | CCG to CAG | GCT to GAT | |

In one embodiment of the present invention, the calculation of information type ii) of step c) involves the calculation of the relative contribution of one or more of the 96 base substitution classes as defined above using either the pyrimidine or the purine of the germline Watson-Crick base pair, such as for example the relative contribution of each of the 96 base substitution classes as defined for either DNA or RNA above using either the pyrimidine or the purine of the germline Watson-Crick base pair,

Single base substitution may occur in wide variety of genomic regions, and may therefore be found in a range of various DNA sequences. In one embodiment of the present invention, the single base substitution frequency (information types i) and/or ii) of step c) is calculated by taking into account all single base substitutions in the genome of a tumour. In another embodiment, the single base substitution frequency (information types i) and/or ii) of step c) is calculated by taking into account all single base substitutions in the genome of a tumour that can be detected using the available data.

In other embodiments of the present invention, the single base substitution frequency (information types i) and/or ii) of step c) is calculated by using all single base substitutions in specific encoding regions, genes and/or exons in the genome of a tumour.

### Information On Copy Number Variation for Use in the Method of the Invention

Genomic regions may be structurally altered in cancer, thus resulting in genomic regions that differ in copy number from the copy number of the same genomic region in a healthy cell or a non-cancerous cell. In the case of most genes, the copy number of a healthy cell or a non-cancerous cell is normally 2, since the gene is present on two chromosomes. For such genes, the presence of more than 2 or less than 2 copies of the gene is a sign of copy number variation, which may be caused by cancer, In one embodiment of the present invention, the copy number of genomic regions and/or genes in biological sequences of said bodily sample is compared to the copy number of the corresponding genomic regions and/or genes in a normal sample of healthy or non-cancerous material and used for calculating the classification score.

The CNV status of a genomic region, gene (information type iii) can be determined by hybridization-based methods, including SNP array, CGH array, spectral karyotyping, FISH or by sequencing based methods in which the relative sequencing depth is analyzed, for example as described by Favero et al 2014, or by gene expression profiling, and other methods commonly used in the art for determination of copy number.

Information of the CNV status of a genomic region, gene or exome (information type iii) of step c) can be encoded in different ways to correlate with the copy number variation of a given genomic region, gene or set of genes in a sample and used for calculation of the classification score in a method according to the invention. For example, the CNV status (information type iii) of step c) may be encoded as altered (1) or non-altered (0) compared to a normal sample including the same genomic region, gene or set of genes in healthy tissue or non-cancerous tissue and used in the present method, alternatively the CNV information can be encoded as −1, 0 or +1, corresponding to a copy number of <2, 2 or >2 if the chromosome is an autosome, or <1, 1, or >1 if the chromosome is a sex chromosome, or the CNV information can be encoded as consisting of the specific copy number of a genomic region, gene or set of genes, or the CNV information can be encoded as the consisting of difference in the copy number of a genomic region, gene or set of genes compared to the same genomic region, gene or set of genes in healthy tissue or non-cancerous tissue.

The copy number variation of different specific genes may be associated with different cancer types. In one embodiment of the present invention, the calculation of copy number variation (information type iii) of step c) is based on the copy number variation of genes which are mutated in cancer, such as determined by using a method which involves identifying genes that are mutated more frequently than expected from the background mutation rate in any individual cancer type, as identified through genomic analysis of several individual tumours as described by Lawrence et al 2013. In one embodiment of the present invention, the copy number variation is derived in genes that are mutated in cancer, wherein the selected genes are mutated more frequently than expected from the background mutation rate in any individual cancer type, as identified through genomic analysis of several individual tumours.

In a more preferred embodiment of the present invention, the method calculates a classification score using copy number variation (information type iii) of step c) in a set of genes encoding or corresponding to the sequences of SEQ ID NO: 1 to 231.

### Classifiers for Use in the Methods of the Invention

One advantage of using classifiers based on machine-learning methods is that such methods can be used and trained to take into account complex relations in the input data, such as for example statistical interactions between mutations in different genes, which are not easily described by simple rules. This can potentially result in better predictive performance.

In an embodiment of the current invention, the method is computer-implemented and involves the use of at least one classifier, or a plurality of classifiers that is based on a machine learning method.

Examples of machine learning methods that may be used for the method according to the present invention can include the following: artificial neural network, backpropagation, boosting, bayesian statistics, decision tree learning, Gaussian process regression, kernel estimators, naive Bayes classifier, nearest neighbor algorithm, restricted Boltzmann machine, stepwise additive logistic regression, support vector machines, random forests, ensembles of classifiers,.

In a more preferred embodiment, the method according to the present invention is computer-implemented and involves the use of at least one classifier or a plurality of classifiers each being based on a machine learning method selected from the group consisting of decision trees, random forests, stepwise additive logistic regression, artificial neural networks and support vector machines.

In an even more preferred embodiment of the present invention, the method according to the present invention is computer-implemented and involves the use a random forest classifier.

### Calculation of Classification Score and Ranking of the Cancer Types

The method according to the present invention produces a ranking of a plurality of cancer types based on the classification score calculated by use of mutation status of step b) in combination with one or more of information types i) to iii) of step c) of the method.

In one embodiment of the present invention, said classification score for each of a plurality of cancer types in a subject is calculated as the proportion of classifiers that predict a given type of cancer.

In a more preferred embodiment of the present invention, the method involves the use of a random forest classifying method and the classification score for a given type of cancer is calculated as the proportion of trees that predict the given type of cancer.

According to the method of the present invention, the plurality of cancer types is ranked based on their likelihood of being present in a sample. Such a ranking may preferably be performed by listing the cancer types based on classification score and by descending classification score. When such a ranked list is used for predicting a specific type of cancer in a subject, the highest ranking cancer types, most preferably the top ranking cancer type, the top two ranking cancer types or the top three ranking cancer types may be used for predicting the cancer type in a subject, and selecting further clinical test to be performed on said subject.

### Confidence Scores for Use in the Method of the Invention

In some cases, the differences in classification score calculated for two cancer types among the plurality of cancer types may be minor. In such cases, the confidence of a prediction of a given type of cancer based on the ranking of classification scores may be reduced, and the identity of the cancer type ranking number two (i.e. the cancer type with second highest classification score) may be useful for clinical testing as well, since there is an increased chance that the primary tumour may originate from that cancer tissue type.

In one embodiment of the present invention, the method further comprises a step wherein a confidence score is calculated as the difference between the classification scores from the two highest ranking types of cancer. The authors have found that such a confidence score helps to point to specific subjects, wherein the top two or three ranking cancer types should be taken into account and used in the prediction of cancer type in a subject.

### Cancer Types Predicted By the Method of the Invention

Cancer (malignant neoplasm) is a class of diseases in which a group of cells display the traits of uncontrolled growth (growth and division beyond the normal limits), invasion (intrusion on and destruction of adjacent tissues), and sometimes metastasis (spread to other locations in the body via lymph or blood). These three malignant properties of cancers differentiate them from benign tumours, which are self-limited, do not invade or metastasize. Most cancers form a tumour but some, like leukemia, do not.

Cancers are classified by the type of cell that resembles the tumour and, therefore, the tissue presumed to be the origin of the tumour. The following general categories are applied:

Carcinoma: malignant tumours derived from epithelial cells. This group includes the most common cancers, comprising the common forms of breast, prostate, lung and colon cancer.

Lymphoma and Leukemia: malignant tumours derived from blood and bone marrow cells.

Sarcoma: malignant u ours derived from connective tissue, or mesenchymal cells

Mesothelioma: tumours derived from the mesothelial cells lining the peritoneum and the pleura.

Glioma: tumours derived from glia, the most common type of brain cell. Germinoma: tumours derived from germ cells, normally found in the testicle and ovary.

Choriocarcinoma: malignant tumours derived from the placenta.

The method of the present invention is useful for prediction of a cancer type among a plurality of different cancer types, such as for example one or more cancers selected from the group consisting of carcinoma, lymphoma, leukemia, sarcoma, mesothelioma, glioma, germinoma and choriocarcinoma.

In one embodiment, the cancer is a non-CNS (Central Nervous System) cancer.

Examples of cancers for which the method according to the present invention can calculate classification scores include: colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumour, cervical cancer, testicular tumour, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumours, liver cancer, laryngeal cancer, esophageal cancer, mammary tumours, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumours, endometrial cancer, gastric cancer, cancer of the head and neck, chronic lymphoid leukemia, hairy cell leukemia and thyroid cancer

In one preferred embodiment of the present invention, the plurality of cancer types for which the method of the present invention calculates classification scores comprises at least the following types of cancer: breast, endometrium, kidney, large intestine, liver, lung, ovary, pancreas, prostate, and skin cancer. In a more preferred embodiment, the plurality of cancer types for which the method of the present invention calculates classification scores consists of the following types of cancer: breast, endometrium, kidney, large intestine, liver, lung, ovary, pancreas, prostate, and skin cancer.

In another preferred embodiment of the present invention, the plurality of cancer types for which the method of the present invention calculates classification scores comprises at least the following types of cancer: breast, endometrium, kidney, large intestine, lung and ovary cancer. In an even more preferred embodiment, the plurality of cancer types for which the method of the present invention calculates classification scores consists of the following types of cancer: breast, endometrium, kidney, large intestine, lung and ovary cancer.

A bodily sample according to the present invention can be any type of bodily sample which may include cancer material such as cancer cells, DNA, RNA or protein. In one preferred embodiment of the present invention, the bodily sample comprises tumour cells or tumour DNA.

Examples of acquired bodily samples which may be used in the method according to the present invention include bodily tissue samples and/or bodily fluid samples. The type of bodily sample used and method for acquiring such bodily samples may be varied based on the type of cancer.

Tumours located in a variety of organs may shed cells or mutated DNA into bodily fluids such as e.g. the bloodstream which gives rise to circulating tumour DNA (ctDNA) or circulating tumour cells. This phenomenon allows for the use of bodily fluid samples which are acquired by use of minimally invasive or non-invasive methods for predicting cancer in methods as disclosed herein. The use of such bodily fluid samples has many advantages, one is that the subject is spared the pain of obtaining a bodily sample by use of an invasive method. Another is that such bodily fluids may be obtained more frequently, and this allows for the use of the method according to the present invention for screening of a larger population of subjects for the presence of cancer.

Samples of bodily fluids which may comprise cancer cells or cancer DNA according to the present invention may include amniotic fluid, aqueous humour and vitreous humour, bile, blood, serum, plasma, breast milk, cerebrospinal fluid, cerumen (earwax), chyle, chime, endolymph and perilymph fluid, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion and vomit.

Tumours located in variety of organs may shed cells or mutated DNA into bodily fluids such as e.g. the bloodstream. In a preferred embodiment of the present invention, the bodily sample is a sample of bodily fluid comprising circulating tumour cells or circulating tumour DNA (ctDNA), such as blood, serum, plasma, urine, lymph fluid, sputum or bronchial washing fluid.

Bodily tissue samples may be acquired by performing biopsy, surgery, scraping or other commonly known methods for acquiring bodily tissue samples. In a preferred embodiment of the present invention, the bodily sample is acquired by performing biopsy or surgery.

In cases wherein a subject presents with metastatic cancer and wherein the primary cancer tissue type is not known, the method of the present invention can be used to predict the primary cancer tissue based on an acquired sample comprising metastatic cancer material. Thus in a preferred embodiment of the present invention, the acquired bodily sample comprises metastatic cancer material, such as for example a biopsy of a metastasis or a sample of bodily fluid comprising metastatic cancer material.

A representative way of performing diagnoses of the origin of a tumour is illustrated in the flow chart of FIG. 8. When a new patient with a tumour is seen in the clinic, the most likely outcome it that the tumour is a primary tumour and thus that the origin is evident. In approximately 10% of all cases, the tumour causing symptoms and thus detected first is a metastasis from a tumour in another tissue. In about half of the cases of metastatic cancer, the primary tumour is readily located, while in the other half of cases, the primary tumour is not readily located. These are known as metastases of unknown origin (MUO). Additional diagnostic tests are employed to detect the origin of the MUO but in about 2-4% of all cases, the primary origin is not located. These are known as cancers of unknown primary (CUP) and are the most difficult cases to treat. By using the methods disclosed herein the percentage of MUO and CUP can be reduced. Additionally, the number of diagnostic procedures can be reduced or focused on the most likely origin.

The method according to the present invention may be computer implemented and performed by use of different entities. For example, one or more steps of the method may be performed on a client, which may then send information derived from or calculated in the various steps of the method to a server. Thus, in one embodiment of the method of the invention at least one of steps a) to c) is performed by a client, wherein said client is capable of sending to a server one or more types of data according to claim 1 selected from the group consisting of genomic profile, mutation status, information type I), information type ii) information type iii), classification score and ranking.

In another embodiment of the of the method of the invention at least one of steps b) to e) is performed by a server, wherein said server is capable of receiving from a client one or more types of data according to claim 1 selected from the group consisting of genomic profile, mutation status, information type i), information type ii) information type iii), classification score and ranking.

### Products for Performing the Method of the Invention

Another aspect of the present invention is to provide a computer program product having instructions which when executed by a computing device or system causes the computing device or system to carry out the method according to the invention as described herein.

Still another aspect of the invention is to provide a data-processing system having means for carrying out the method according to the invention as described herein.

Still another aspect of the invention is to provide a computer readable medium having stored there on a computer program product having instructions which when executed by a computing device or system causes the computing device or system to carry out the method according to the invention as described herein.

### Items

The following set of items further describes the invention:

Item:
1. A method for prediction of a specific type of cancer in a subject using an acquired bodily sample from said subject, said method comprising the steps of:
   a) providing biological sequences derived from said bodily sample,
   b) deriving the mutation status of specific genes that are mutated in cancer from said biological sequences compared to a normal sample,
   c) calculating one or more of the following types of information i) to iii) from said biological sequences:
      i) single base substitution frequency wherein the identity of the two bases flanking said substitution is not taken into account,
      ii) single base substitution frequency in triplets of nucleotide bases, wherein the identity of the two bases flanking the substitution is taken into account,
      iii) copy number variation (CNV) of genomic regions and/or genes compared to the copy number of the same regions and/or genes in a normal sample
   d) calculating a classification score for the presence of each of a plurality of cancer types in said subject, wherein said classification score calculation is based on the mutation status derived from step b) in combination with the one or more of the information types i) to iii) being calculated in step c),
   e) ranking the plurality of cancer types based on the classification score of step d), and
   f) predicting the specific type of cancer in said subject based on the ranking of step e).
2) The method according to item 1, wherein the method calculates said classification score based on a combination of the mutation status derived from step b) in combination with information types i) and/or ii) of step c).
3) The method according to the preceding items, wherein the method calculates said classification score based on a combination of the mutation status derived from step b) in combination with information type ii) of step c).
4) The method according to the preceding items, wherein the method calculates said classification score based on a combination of the mutation status derived from step b) in combination with information type iii) of step c).
5) The method according to the preceding items, wherein the method calculates said classification score based on a combination of the mutation status derived from step b) in combination with information type iii) and one or more of the information types i) and ii) of step c),
6) The method according to the preceding items, wherein the method calculates said classification score based on a combination of the mutation status derived from step b) in combination with information types iii) and ii) of step c),
7) The method according to the preceding items, wherein the method calculates said classification score based on a combination of the mutation status derived from step b) in combination with information types iii) and i) of step c).
8) The method according to the preceding items, wherein said biological sequence is a DNA, mRNA or protein sequence obtained from said bodily sample.
9) The method according to the preceding items, wherein said biological sequence is a DNA and/or mRNA sequence obtained from said bodily sample.
10) The method according to the preceding items, wherein both synonymous and non-synonymous mutations are used for deriving said mutation status.
11) The method according to the preceding items, wherein only non-synonymous mutations are used for deriving said mutation status.
12) The method according to the preceding items, wherein the mutation status of step b) is based on mutation status of genes that are recurrently mutated in association with cancer.
13) The method according to the preceding items, wherein the mutation status of step b) is calculated in a set of genes encoding the sequences of SEQ ID NO: 1 to 231 and/or in the sequences of SEQ ID NO: 1 to 231.
14) The method according to the preceding items, wherein the calculation of information types i) of step c) is encoded as the relative contribution for one or more of the 12 substitution classes.
15) The method according to the preceding items, wherein the calculation of information types i) and ii) of step c) is only based on the pyrimidine of the germ-line Watson-Crick base-pair and the single base substitutions are selected from the group consisting of C to A, C to G, C to T, T to A, T to C and T to G.
16) The method according to the preceding items, wherein the calculation of information types i) and ii) of step c) is only based on the purine of the germ-line Watson-Crick base-pair and the single base substitutions are selected from the group consisting of A to C, A to T, A to G, G to T, G to C and G to A.
17) The method according to items 12 and 13, wherein the calculation of information types i) of step c) is encoded as the relative contribution for each of the possible 6 substitution classes
18) The method according to the preceding items, wherein the calculation of information types ii) of step c) is calculated based on the pyrimidine of the germ-line Watson-Crick base-pair and the single base substitutions are selected from the group consisting of C to A, C to G, C to T, T to A, T to C and T to G, and wherein said single base substitution frequency in triplets for each member of this group is calculated by using the number of each specific type of single base substitution in triplets of nucleotide bases.
19) The method according to the preceding items, wherein the calculation of information types ii) of step c) is calculated based on the purine of the germ-line Watson-Crick base-pair and the single base substitutions are selected from the group consisting of A to C, A to T, A to G, G to T, G to C and G to A, and wherein said single base substitution frequency in triplets for each member of this group is calculated by using the number of each specific type of single base substitution in triplets of nucleotide bases.
20) The method according to items 15 to 16, wherein the calculation of information types ii) of step c) is encoded as the relative contribution for each of the possible 96 classes.
21) The method according to the preceding items, wherein information types i) and/or ii) of step c) are calculated based on substitutions in biological sequences encoded by the genome of said subject.
22) The method according to the preceding items, wherein information types i) and/or ii) of step c) are calculated based on substitutions in biological sequences in specific genes of said subject.
23) The method according to the preceding items, wherein information types i) and/or ii) of step c) are calculated based on substitutions in biological sequences in specific encoding regions, genes and/or exons.
24) The method according to the preceding items, wherein the calculation of information type iii) of step c) is encoded to correlate with the copy number variation of genomic regions, genes and/or sets of genes.
25) The method according to the preceding items, wherein the calculation of information type iii) of step c) is encoded as −1, 0 and +1, corresponding in autosomes to a copy number of <2, 2 and >2 respectively, or in sex chromosomes to a copy number of <1, 1 and >1 respectively.
26) The method according to the preceding items, wherein the calculation of information type iii) of step c) consists of the copy number or the difference in copy number of genomic regions, genes and/or sets of genes in said sample compared to the copy number of the same genomic regions, genes and/or sets of genes a normal sample.
27) The method according to the preceding items, wherein the calculation of information type iii) of step c) is based on the copy number variation of genes associated with cancer.
28) The method according to the preceding items, wherein the calculation of information types iii) of step c) is based on the copy number variation of a set of genes encoding the sequences of SEQ ID NO: 1 to 231.
29) The method according to any of the preceding items, wherein the calculation of information types i) to iii) of step c) and/or the mutation status of step b) is based on a comparison to a normal bodily sample from the same subject.
30) The method according to any of the preceding items, wherein said method is computer-implemented and involves the use of at least one classifier that is based on a machine learning method, preferably selected from the group consisting of decision trees, random forests, stepwise additive logistic regression, artificial neural networks and support vector machines, and more preferably wherein the machine learning method is random forests.
31) The method of item 30 wherein said method involves the use of a plurality of classifiers which are based on a machine learning method, preferably selected from the group consisting of decision trees, random forests, stepwise additive logistic regression, artificial neural networks and support vector machines, and more preferably wherein the machine learning method is random forests.
32) The method according to any of the preceding items, wherein said method is computer-implemented and uses a random forest classifying method.
33) The method according to any of the preceding items, wherein the method takes into account statistical interactions between mutations.
34) The method according to any of the preceding items, wherein the plurality of cancer types comprises at least the following types of cancer: breast, endometrium, kidney, large intestine, liver, lung, ovary, pancreas, prostate, and skin cancer.
35) The method according to any of the preceding items, wherein the plurality of cancer types comprises at least the following types of cancer: breast, endometrium, kidney, large intestine, lung and ovary cancer.
36) The method according to any of the preceding items, wherein the plurality of cancer types consists of the following types of cancer: breast, endometrium, kidney, large intestine, liver, lung, ovary, pancreas, prostate, and skin cancer.
37) The method according to any of the preceding items, wherein the plurality of cancer types consists of the following types of cancer: breast, endometrium, kidney, large intestine, lung and ovary cancer.
38) The method according to any of the preceding items, wherein said ranking is based on a sorted list of classification scores from one or more classifiers,
39) The method according to any of the preceding items, wherein the classification score for each cancer type is calculated as the proportion of positive-voting trees in a random forests classifier trained to distinguish that cancer type from all other cancer types.
40) The method according to any of the preceding items, further comprising a step wherein a confidence score is calculated as the difference between the classification scores from the two highest ranking types of cancer.
41) The method according to any of the preceding items, wherein the bodily sample is a bodily tissue sample or a bodily fluid sample.
42) The method according to any of the preceding items, wherein the bodily sample is a bodily fluid sample such as sample of blood, serum, plasma, urine, lymph fluid, sputum or bronchial washing fluid.
43) The method according to any of the preceding items, wherein said sample comprises tumour cells or tumour DNA.
44) The method according to any of the preceding items, wherein the bodily sample is a sample comprising circulating tumour DNA (ctDNA) or circulating tumor cells.
45) The method according to any of items 1-42, wherein said bodily sample comprises metastatic cancer material, such as a biopsy sample of a metastasis or a sample of bodily fluid comprising metastatic cancer material.
46) The method according to any of the preceding items, wherein at least one of steps a) to c) is performed by a client, and wherein said client is capable of sending to a server one or more types of data according to item 1 selected from the group consisting of genomic profile, mutation status, information type i), information type ii) information type iii), classification score and ranking.
47) The method according to any of the preceding items, wherein at least one of steps b) to e) is performed by a server, and wherein said server is capable of receiving from a client one or more types of data according to item 1 selected from the group consisting of genomic profile, mutation status, information type i), information type ii) information type iii), classification score and ranking.
48) A computer program product having instructions which when executed by a computing device or system causes the computing device or system to carry out the method according to any one of items 1 to 47.
49) A data-processing system having means for carrying out the method according to any one of items 1 to 47.
50) A computer readable medium having stored thereon a computer program product according to item 48.

### EXAMPLES

### Example 1

The present example describes the development and testing of prediction methods as described herein.

### Data

We used the publically available COSMIC Whole Genomes database to identify tumour specimens with genome-wide or exome-wide somatic mutation data, and focused on solid non-central nervous system (non-CNS) tumours of the ten primary cancer tissue sites for which at least 200 unique specimens were available (Table 3).

TABLE 3
Total number of samples with mutation data representing each
cancer tissue type, and the number that also has CNV data,
including those in the training set and those in the testing set

Number of samples
with data for:

| Table 3 | Point | Copy number |
|---|---|---|
| Primary site | mutations | variations |
| Breast | 936 | 850 |
| Endometrium | 281 | 246 |
| Kidney | 468 | 300 |
| Large Intestine | 592 | 486 |
| Liver | 415 | — |
| Lung | 807 | 476 |
| Ovary | 497 | 462 |
| Pancreas | 311 | — |
| Prostate | 372 | — |
| Skin | 296 | — |
| Total | 4975 | 2820 |

Somatic mutation data from the COSMIC database version 68 by Bamford et al. was downloaded at Feb. 8, 2014 (ftp://ftp.sanger.ac.uk/pub/CGP/cosmicidata_export/CosmicMutantExport_v68.tsv.gz),

The downloaded data corresponded to 235589 samples. Out of these, 227757 samples were removed, which were samples that were not labeled as “Genome:wide.screen” (227512 samples), and samples labeled as cell-line (5064 samples).

Furthermore, in ten cases, two sample IDs matched to the same tumour ID, meaning one tumour gave rise to two samples in the data set. In 105 cases, the same sample name matched to more than one tumour ID. Samples were removed to leave only one sample per tumour ID. When deciding which sample to keep, the following priorities were made: Surgery biopsy, primary, verified and exome seq had priority over xenograft, relapse, unverified and RNA-Seq, respectively.

Gene annotation in the database was not entirely consistent and thus required additional curation. We mapped as many genes as possible to Ensembl gene IDs, by searching for gene information in the following columns: Accession.Number, HGNC.ID. and Gene.name, which in most cases contained the gene symbol, but was also found to hold Ensembl gene IDs and Swissprot accession numbers. We were able to annotate Ensembl gene IDs to 99,4% of the mutations in COSMIC, Finally, mutations in COSMIC are reported for all possible trancripts, so we filtered the mutation table so that each row corresponded to a single unique mutation identified by its genomic position.

We also downloaded all available CNV data from the COSMIC database at Feb. 8, 2014 (ftp://ftp.sanger.ac.uk/pub/CGP/cosmicidata_export/CosmicCompleteCNV_v68.tsv.gz), and mapped the genes overlapping with each CNV segment.

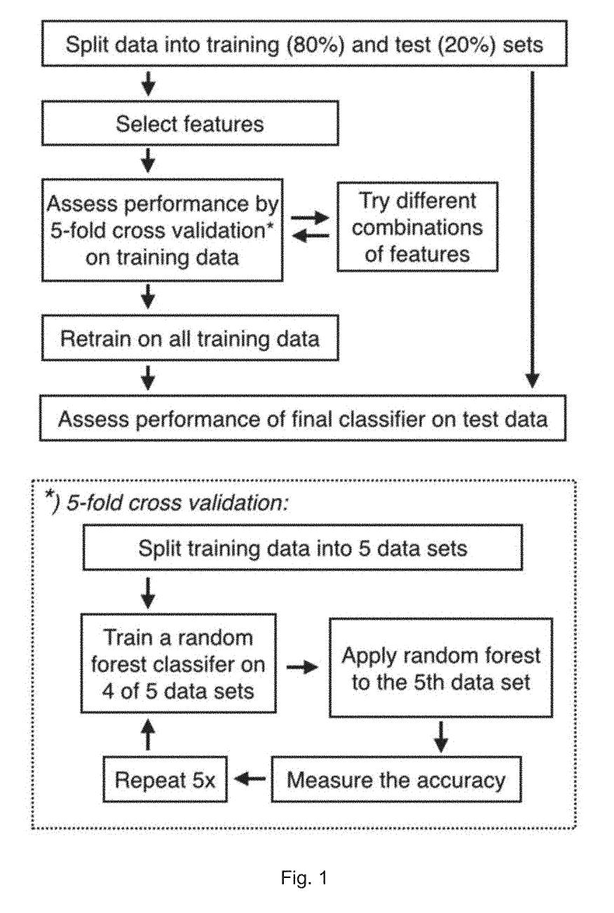

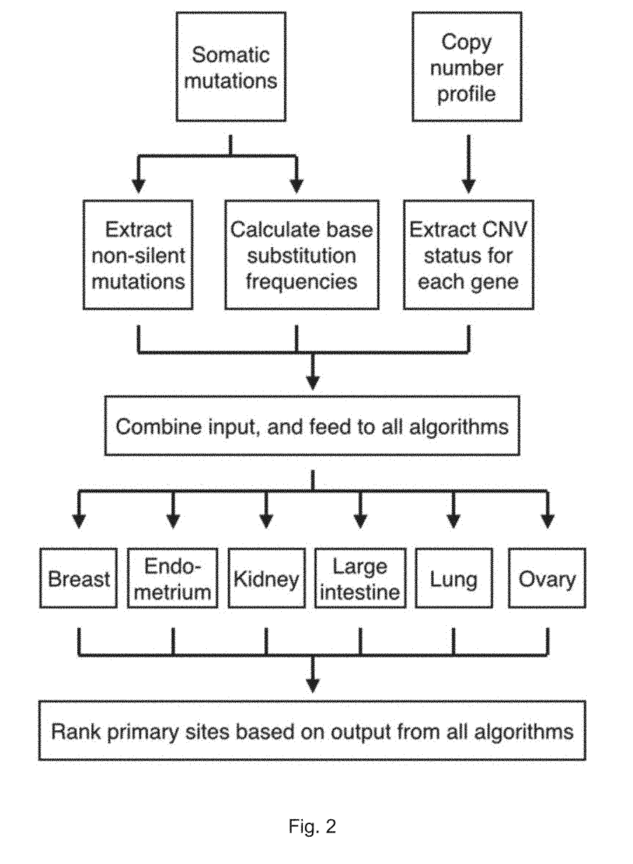

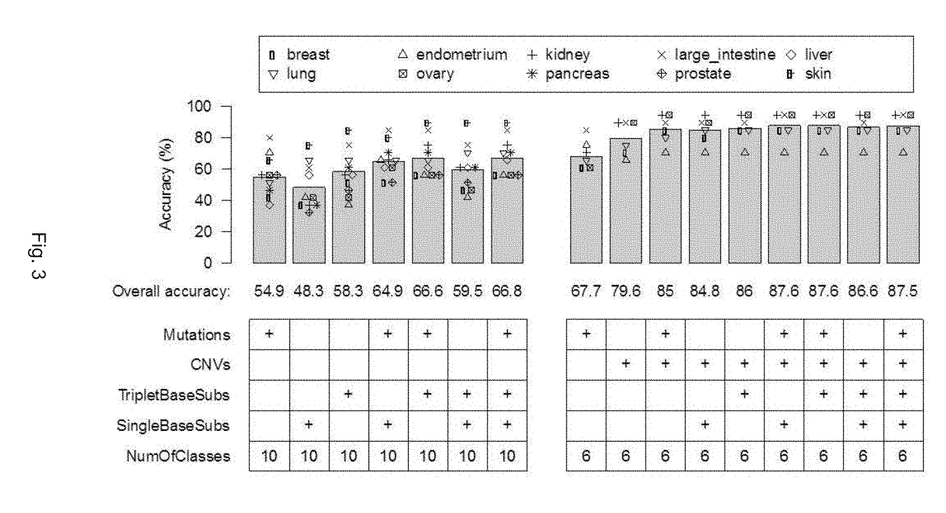

The resulting ˜5000 specimens were split randomly, while retaining proportionality of each class, into a training set of ˜4000 specimens used to derive a classifier, and a validation set of ˜1000 that was not used except to evaluate the final classifier. We used five-fold cross validation on the training set to select the machine learning method and features as described below (FIG. 1).

### Evaluation of Classifier Performance

We trained a set of random forest classifiers to identify each of 10 primary cancer tissue sites from the chosen information input variables. Selection of features (i.e. which types of input should be used in methods for prediction of cancer) was performed using 5-fold cross-validation: the training data was split into five parts, conserving the distribution of the ten primary cancer tissue sites within each part. For each primary cancer tissue site, we trained a random forest on four parts combined, evaluated its performance on the fifth part, and repeated this for all five cross validations. Separate models were trained for each primary site, each learning to distinguish one site from the remaining primary sites.

Performance was evaluated by collecting the classification scores for each primary cancer tissue site, and predicting a primary cancer tissue site for each sample by picking the highest classification score. Accuracy was calculated as the fraction of samples across all cross-validation test sets that were correctly classified by the first proposal. After input information features were selected, they were used to train a final model, using the entire training data set. Performance of the final models was evaluated using the test set that was set aside prior to cross-validation.

### Selection of Machine Learning Method

We tested four commonly used machine learning methods: stepwise additive logistic regression, artificial neural networks, support vector machines, and random forests.

For each machine learning method, we trained a set of ten classifiers, using input information of the mutation status of 231 genes mutated in cancer in combination with calculated single base substitution frequency (without taking into account the identity of the two flanking bases and calculated using only the pyrimidine of the germ line Watson-Crick base pair). Each classifier was trained to discriminate one primary cancer tissue site from the other nine, using the input of 236 features.

Random forest classifiers based on Breiman (2001) were trained using the randomForest by Liaw and Wiener (2002) package v.4.6-7 in R, using the default parameters to grow 500 trees, and sample \sqrt{p} features as candidates at each split within a tree, where p is the total number of features. Stratified sampling was used to draw equal numbers of cases and non-cases for each tree, with sample size equal to 0.632 times the size of the smallest group. When applied to a new data sample, we define the “classification score” as the proportion of the trees that voted for the given primary site.

Based on cross-validation accuracy, we found that the method (using input information of the mutation status of 231 genes mutated in cancer in combination with calculated single base substitution frequency (without taking into account the identity of the two flanking bases and calculated using only the pyrimidine of the germ line Watson-Crick base pair)) worked successfully with all the different classifiers which were tested, however the use of a random forest classifier provided the best performance in 9 out of 10 primary sites (FIG. 6).

### Selection of Input Information for the Method

We next aimed to identify a set of features (i.e. a set of input information types) for our method, derived from the mutation data that could most accurately identify the primary cancer tissue site of a tumour. We used 5-fold cross validation to assess the classification accuracy using various combinations of the following sets of features:

### Mutation Status of Recurrent Cancer Genes

We chose a previously published list of 231 genes that are recurrent in cancer (Lawrence et al. 2014) (see Table 1) and counted the number of non-synonymous mutations within the coding region (or exomes) of each gene. When training a model with these features alone we achieved a cross-validation accuracy of 55% across the ten primary sites (FIG. 3). Accuracy varied among primary sites, from 36% for liver to 78% for large intestine.

### Single Base Substitution Frequency

Single base substitutions are found at different frequencies across tumours, likely reflecting the mutational processes that shaped the tumour genome. For each tumour sample, we used all base substitution mutations, regardless of their effect, to calculate the relative frequencies of the six different classes of single base substitutions based on the pyrimidine of the Watson-Crick germ-line base-pair. Base substitutions alone classified primary cancer tissue site with an overall accuracy of 48% (range=30-69%), but when combined with the mutational variables described above accuracy increased to 65% (range=51-84%) (FIG. 3).

### Trinucleotide-Context Base Substitution Frequency

For each tumour sample, we used all base substitution mutations to calculate the relative frequencies of the 96 possible single base substitutions in trinucleotides included in Table 2 herein. Single base substitutions frequencies in trinucleotides alone identified primary cancer tissue site with an overall accuracy of 58% (range=39-86%), but when combined with the mutational status variables described above accuracy increased to 66% (range=54-90%) (FIG. 3).

### Position-Specific Mutations

Of the many mutations that could occur within a gene, some may be selected for or against in different tissues, depending on which signaling pathways are active in the pre-malignant cell. Therefore, mutations at certain positions may be specific to certain cell types or primary sites; this has been described for EGFR (Lawrence et al. 2014). We identified a set of mutation hotspots, positions or gene regions that were significantly more frequently mutated in our training data than expected by chance. Addition of these hotspot features to the model using all non-synonymous mutation status within the set of 231 genes as denoted in Table 1 had no effect on accuracy (data not shown).

We next considered whether copy number profiles could improve classification performance. However, CNV data was available for only ˜40% of the samples in the COSMIC Whole Genomes database. Thus, we assessed the performance of classifiers using CNV data in a separate analysis, reducing the number of samples and thereby also the number of primary cancer tissue sites from ten to six. This increases the expected accuracy of a random classifier from 1/10=10% to 1/6=17%, and so for proper comparison we repeated some of the previous analyses on the reduced data set. In this reduced data set, non-synonymous mutation status of exons in the 231 specific genes of Table 1 mutated in cancer alone classified primary site with an accuracy of 69% (FIG. 3),

Each gene that was used for derivation of mutation status as described above was also encoded as a copy number variable (loss, gain or normal copy number). Using copy number variables alone resulted in an accuracy of 80%, and when combined with mutation status increased to 85%. Further adding one or both of single base substitution frequencies wherein the flanking bases where not taken into account and trinucleotide frequencies (single substitution frequencies wherein the identity of the two flanking bases are taken into account) increased accuracy to 87-88% (FIG. 3).

### Selection of Features and Performance on Test Data

We used the cross-validation-based results to assess which features to use in a final classifier of primary cancer tissue site. In addition to the 231 genes, with features for their mutation status and where possible copy number status, we found that overall the use of trinucleotide base substitution frequencies provided the highest accuracy (66.6% and 87,6%, without and with CNVs, respectively). We therefore applied these two models (one using mutation status in combination with trinucleotide base substitution frequencies and the other using a combination of mutation status in combination with trinucleotide base substitution and copy number variation) to the fraction of COSMIC data that had been set aside as test data. We achieved an overall accuracy of 69% and 85% without and with CNVs, respectively (FIG. 3).

We noticed that certain pairs of tissues (e.g, breast-ovary, breast-lung, and endometrium-ovary) seem to be frequently confused (Tables 4 and 5), and that the classifiers for these pairs of tissues often produce elevated output prediction scores for both.

Table 4 and 5 show that the two methods have high sensitivity, ranging from 50-91% for the different primary cancer tissue sites, and also good specificity, ranging from 93-99%.

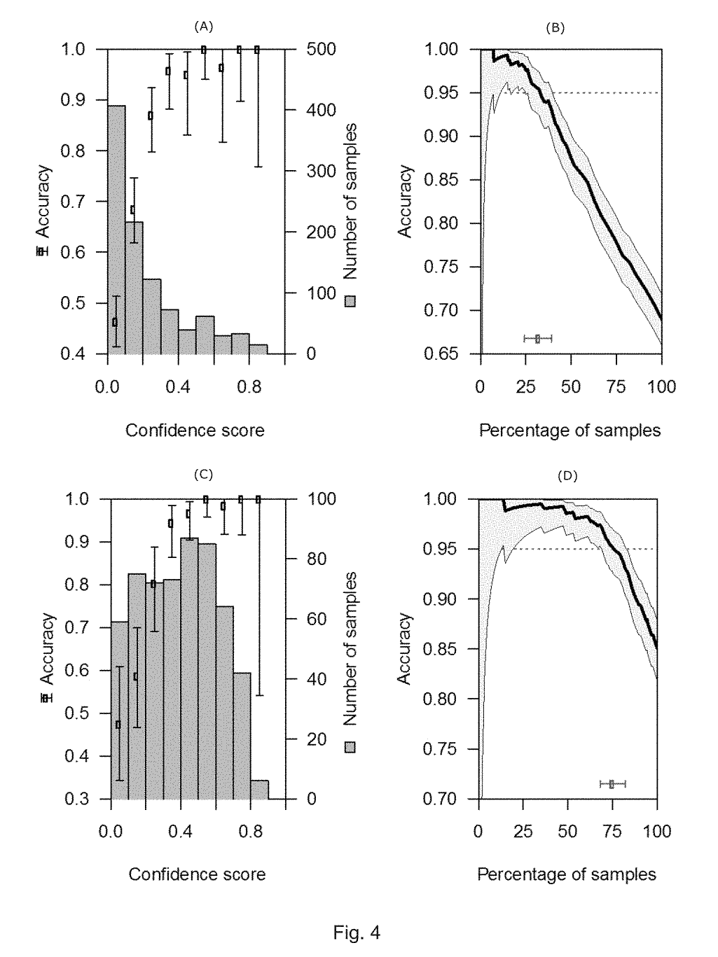

Therefore, we defined a “confidence score” as the difference between the individual classifier output for the two highest-scoring tissues. We found that the confidence score was indeed a strong indicator of accuracy, and that a large fraction of high-confidence samples could be predicted with high accuracy (FIG. 4 and FIG. 7).

TABLE 4
Confusion matrix for the classifier based on mutations status and trinucleotide base substitutions.Total number of samples
per primary cancer tissue site are given to the left and below. Sensitivity and specificity for each primary site is given above
and to the right, respectively.

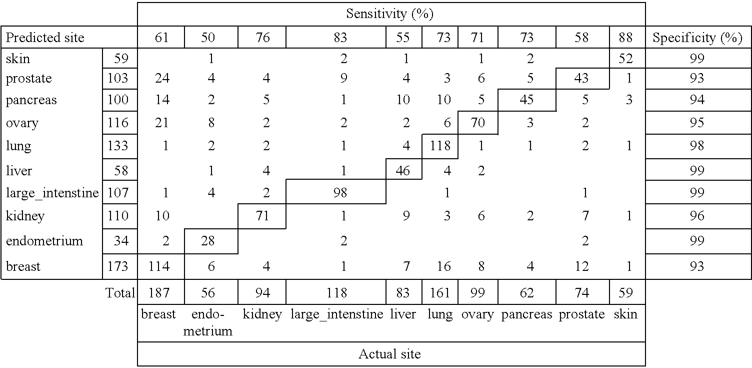

TABLE 5
Confusion matrix for the classifier based on a combination of CNVs, mutation status and
trinucleotide base substitutions. Sensitivity and specificity for each primary cancer tissue site
is given above and to the right, respectively.

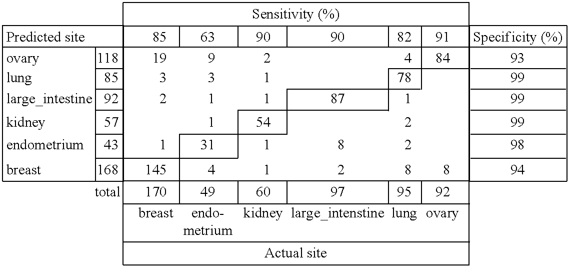

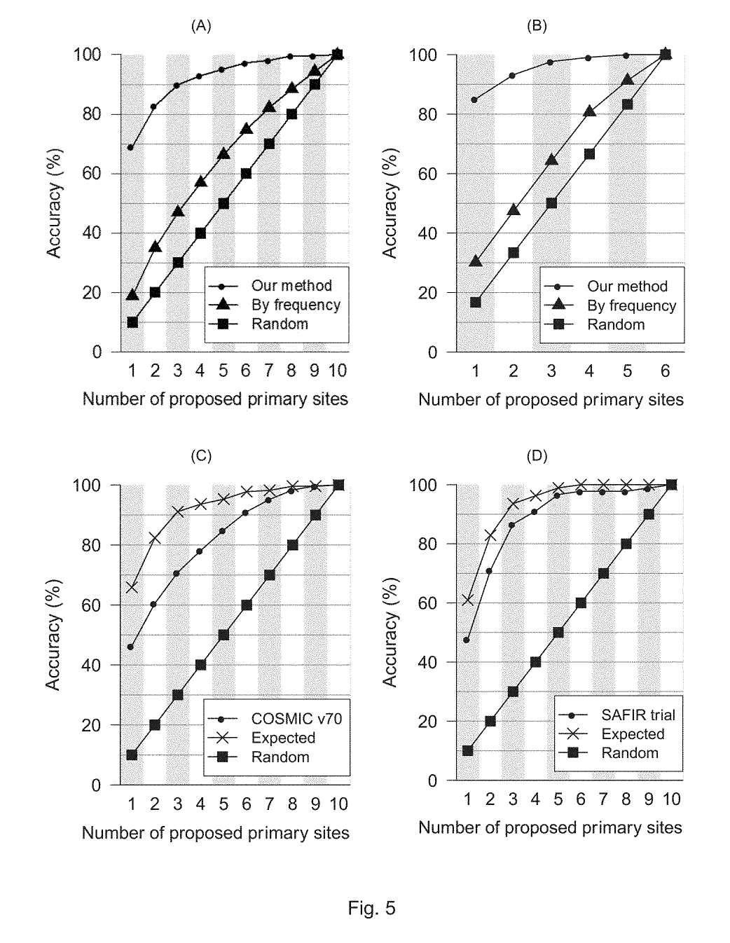

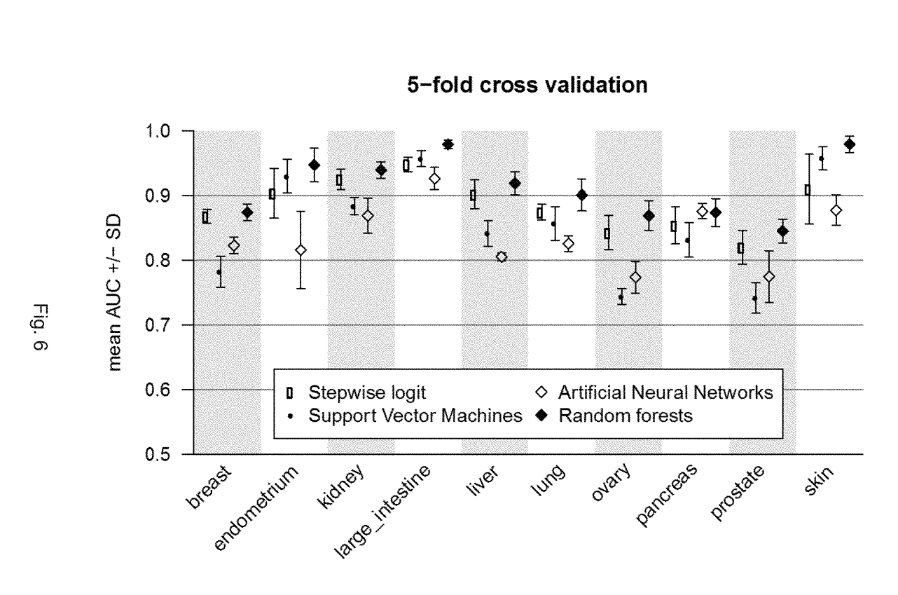

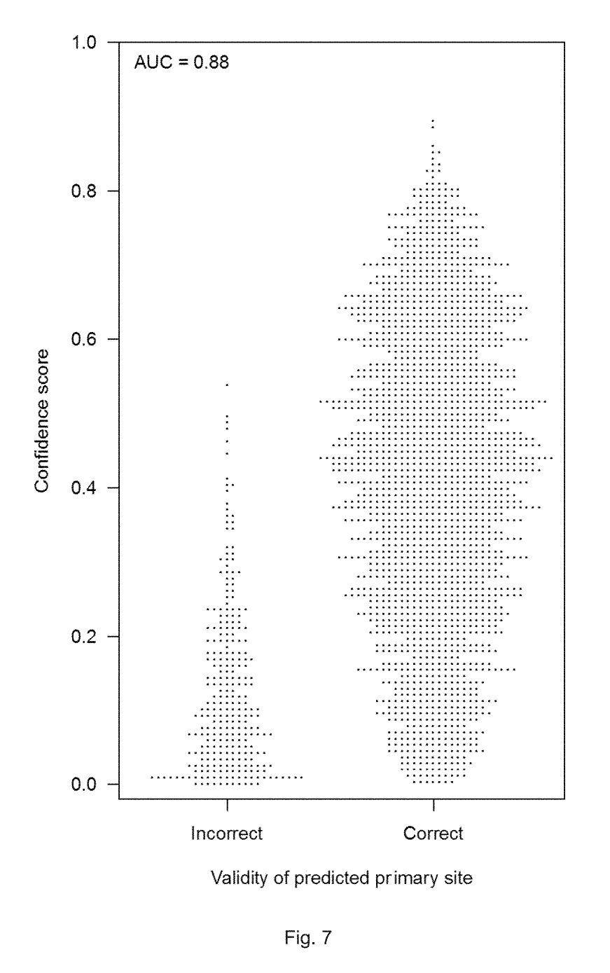

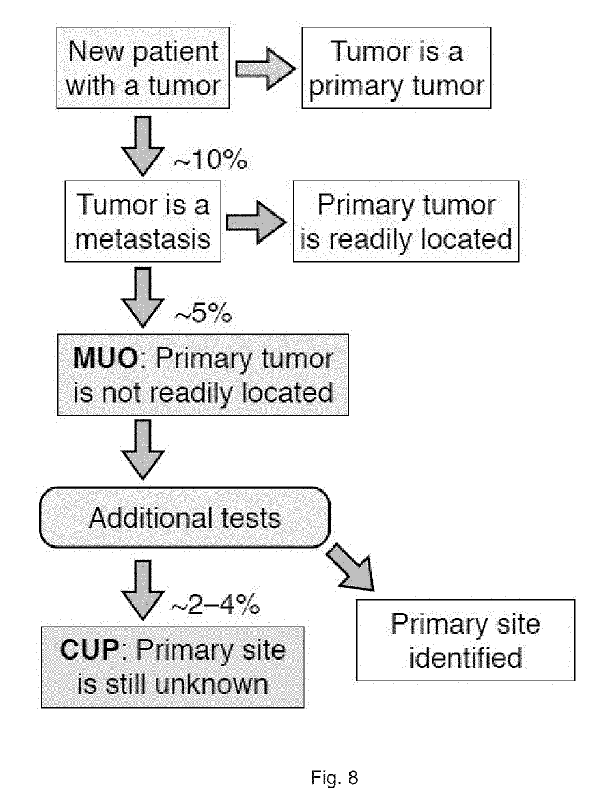

In a clinical application, it would be valuable to produce a ranked list of likely tissues, suggesting the order in which these tissues might be examined in a patient. Thus, we ranked the classification scores of the individual tissue classifiers and assessed the accuracy of the cumulative tissue list; i.e. how frequently the correct tissue is in the top n proposed tissues (FIG. 5). At any number of tissues, our method was substantially more accurate than either random lists or a list of tissues ranked by frequency in the data set.

### Performance on Independent Validation Cohorts

The present section describes the validation of our method using information of mutation status in 231 genes mutated in cancer as described above in combination with trinucleotide single base substitution frequency.

First, we evaluated the performance of our classifier on the latest data from COSMIC. Our model was developed using the data in COSMIC version 68. As an independent test set we downloaded COSMIC version 70, and filtered out any samples that were already entered in v68. All data analysis steps such as quality control, alignment, derivation of mutation status, etc., which could have added a systematic bias, were performed by the authors of the original publications rather than by COSMIC; therefore this data is reasonably independent from the training data.

Since our method predicts the specific cancer types with different accuracy, we calculated the “expected” accuracy of our method for prediction of the types of cancer being present in the validation sets, and compared the predictions of our method in the validation sets to the “expected” accuracy.

The expected accuracy was calculated in the following way:

For each cancer type, m, the number of samples in the validation cohort of this cancer type, Nₘ, was multiplied by the observed sensitivity of our method towards this specific cancer type (see Tables 4 and 5), Sₘ, to give the number of samples of this cancer type expected to be correctly proposed by our method (known as true positives), TPₘ. The overall expected accuracy of our method on the validation cohort is then calculated as the fraction of all expected true positives, TP, out of the total number of samples in the validation cohort, N.

$$\frac{Σ {S}_{m} \cdot {N}_{m}}{N}$$

Sₘ; sensitivity for cancer type m of the TumorTracer method, measured by cross-validation (as found in Table 4 or 5). (number between 0 and 1)
Nₘ: the number of samples in the validation cohort (eg. COSMIC v70, or SAFIR trial) of cancer type m.
N: the total number of samples in the validation cohort

For the COSMIC v70 validation set, the “expected” accuracy was calculated using the numbers of Table 6 below:

TABLE 6

| Cancer type | Nₘ | Sₘ | EXPECTED TP_{M} | |
|---|---|---|---|---|
| Breast | 191 | 61% | 116.51 | |
| Kidney | 240 | 76% | 182.40 | |
| Large intestine | 54 | 83% | 44.82 | |
| Liver | 457 | 55% | 251.35 | |
| Pancreas | 438 | 73% | 319.74 | |
| Prostate | 61 | 58% | 35.38 | |
| Total | N = 1441 | | TP = 950.20 | 950.2/1441 = 65.9% |

Expected accuracy=950.2/1441=0.659

On this independent validation set derived from COSMIC v70, which consisted of 1439 samples from 6 primary cancer tissue sites our model correctly classified the primary cancer tissue site for 46% of the samples (ranging from 25% for kidney to 71% for pancreas) (FIG. 50).

We further applied our classifier to point mutation calls from 91 metastatic breast tumours from SAFIR, a clinical trial to assess benefit of exome sequencing for metastatic breast cancer. Mutation status (also called mutation calls) based on whole exome sequencing data for a cohort of 91 metastatic breast cancers (SAFIR trial) was obtained from the Department of Medical Oncology, Institut Gustave Roussy, Villejuif, France. These calls were derived from whole exome sequencing of tumour and matched normal material following the protocol implemented for the clinical trial. The data did not include information of copy number variation in the metastatic breast tumours.

The “expected” accuracy of the SAFIR validation set was calculated by using the following numbers:

The Safir trial consists of 91 breast cancer samples, and no other cancer types.

Sₘ=61%

Nₘ=91

N=91

Expected accuracy=0.61−91/91=0.61

Note that, because the SAFIR validation set consists of only breast cancer samples, the “expected” accuracy is by definition equivalent to the breast-specific specificity of 61% on the test set (Table 4). Our method using information of mutation status in 231 genes mutated in cancer as described above in combination with trinucleotide single base substitution frequency correctly proposed breast as the primary site in 48% of the samples (FIG. 5D). After breast, the most commonly proposed sites were prostate (18%) and ovary (16%).

FIGS. 5C and D show that the accuracy on our validation data sets is comparable to the test set COSMIC 70. The actual validation accuracy is slightly lower than the “expected” accuracy calculated based on sensitivity of the specific cancer tissue type, this is not surprising due to differences in data generation and analysis. The actual accuracy is still significantly better than a random classifier.

Thus the validation of our method shows that the method performs much better than a random method and with high accuracy.

Comparison with Existing Method

The method disclosed by Dietlein and Eschner 2014 has described a method for predicting the primary cancer tissue site based on mutation status of selected genes (Dietlein and Eschner 2014). In brief, Dietlein and Eschner used mutation data from 905 cell lines originating from 23 different tumour primary sites to select the set of position-specific and nonspecific mutations with the highest discriminatory power for a single primary site. They used this data to train their tool, ICOMS, to infer cancer origin (primary cancer tissue type) from a mutation profile.

The ICOMS method was compared to our method which uses mutation status in combination with single nucleotide substitution in trinucleotides as described above. (This method is called TumorTracer in the below text).

ICOMS was validated on a set of 431 tumours from TOGA, of which 297 were also in the version of COSMIC that we used to develop our method. To provide an unbiased comparison between the two methods, we compared ICOMS prediction calls to TumorTracer prediction calls obtained under cross-validation, and compared both to the actual primary cancer tissue sites.

The distribution of correct and incorrect inferences across the two methods is given in Table 7. Note that the two algorithms deal with uncertainty in different ways: ICOMS in some cases proposes no primary cancer tissue site, whereas TumorTracer always proposes a cancer tissue site along with a corresponding confidence score. Therefore, to do an unbiased comparison of only the high-confidence calls from the two methods, we did a second analysis omitting the lowest confidence proposals by TumorTracer, corresponding to the number of samples for which ICOMS makes no proposal, and compared the performance of each method on the 109 samples for which both methods proposed a primary cancer tissue site (Table 8). Accuracy, defined as the percentage of samples for which the correct primary site was inferred, was significantly higher by TumorTracer than by ICOMS (96% vs. 83%, p=0.003).

TABLE 7
Contingency table with number of tumours correctly
predicted by TumorTracer and by ICOMS. In 129 cases
ICOMS made no primary site diagnosis (labelled “No call”
in the table).

| | | ICOMS | | |
|---|---|---|---|---|
| | | No call | Correct | Incorrect |
| TumorTracer | Correct | 90 | 114 | 28 |
| | Incorrect | 39 | 11 | 15 |

TABLE 8
Contingency table with number of tumours correctly
predicted by TumorTracer and by ICOMS, including
only the 109 samples for which both methods
produced a high-confidence proposal.

ICOMS

| | | Correct | Incorrect |
|---|---|---|---|
| TumorTracer | Correct | 90 | 15 |
| | Incorrect | 1 | 3 |

### CONCLUSION

We developed proof-of-concept classifiers designed to identify the primary cancer tissue site of a tumour from its genomic profile. Specifically, our most accurate classifier used the point mutation status and copy number status of a set of 231 genes recurrently mutated in cancer, as well as the relative frequencies of 96 classes of single base substitutions wherein the identity of the two flanking bases are taken into account. Our single most accurate classifier used the point mutation status of a set of 231 genes recurrently mutated in cancer, in combination with the relative frequencies of 96 classes of single base substitutions wherein the identity of the two flanking bases are taken into account. The latter method was found to have an improved predictive performance compared to a state of the art method for prediction of primary cancer tissue site (ICOMS). As more mutation data becomes available, it will likely be possible to increase accuracy and to develop classifiers for additional tissues, which may involve additional genes.

### REFERENCES

Alexandrov L B, Nik-Zainal S, Wedge D C, et al. (2013a) Signatures of mutational processes in human cancer. Nature 500:415-421. doi: 10.1038/nature12477

Alexandrov L B, Nik-Zainal S, Wedge D C, et al. (2013b) Deciphering Signatures of Mutational Processes Operative in Human Cancer. CellReports 3:246-259. doi: 10.1016/j.celrep.2012.12.008

Altschul S F, Gish W, Miller W, Myers E W, Lipman D J (1990) Basic local alignment search tool. J Mol Biol. 1990 Oct. 5; 215(3):403-10.

Bamford S, Dawson E, Forbes S, et al, (2004) The COSMIC (Catalogue of Somatic Mutations in Cancer) database and website. Br J Cancer. doi: 10.1038/sj.bjc.6601894

Baudis M (2007) Genomic imbalances in 5918 malignant epithelial tumors: an explorative meta-analysis of chromosomal CGH data. BMC Cancer 7:226. doi: 10.1186/1471-2407-7-226

Beroukhim R, Getz G, Nghiemphu L, et al. (2007) Assessing the significance of chromosomal aberrations in cancer: methodology and application to glioma. Proc Natl Acad Sci USA 104:20007-20012. doi: 10.1073/pnas.0710052104

Breiman L (2001) Random forests. Machine learning 45:5-32.

Cibulskis K, Lawrence M S, Carter S L, Sivachenko A, Jaffe D, Sougnez C, Gabriel S, Meyerson M, Lander E S, Getz G. Sensitive detection of somatic point mutations in impure and heterogeneous cancer samples. Nat Biotechnol. 2013 March; 31(3):213-9. doi: 10.1038/nbt.2514. Epub 2013 Feb. 10. PubMed PMID: 23396013; PubMed Central PMCID: PMC3833702.

Chen L L, Blumm N, Christakis N A, et al. (2009) Cancer metastasis networks and the prediction of progression patterns. Br J Cancer 101:749-758. doi: 10.1038/sj.bjc.6605214

Dietlein F, Eschner W (2014) Inferring primary tumor sites from mutation spectra: a meta-analysis of histology-specific aberrations in cancer-derived cell lines. Hum Mol Genet 23:1527-1537. doi: 10.1093/hmg/ddt539

Favero F, Joshi T, Marquard A M, Birkbak N J, Krzystanek M, Li Q, Szallasi Z, Eklund AC (2014) Sequenza: allele-specific copy number and mutation profiles from tumor sequencing data. Ann Oncol. 2014 Oct. 15, pii: mdu479, [Epub ahead of print]

Hess K R, Abbruzzese M C, Lenzi R, et al. (1999) Classification and regression tree analysis of 1000 consecutive patients with unknown primary carcinoma. Clin Cancer Res 5:3403-3410.

Kim S Y, Speed T P (2013) Comparing somatic mutation-callers: beyond Venn diagrams. BMC Bioinformatics 14:189. doi: 10.1186/1471-2105-14-189.

Koboldt D C, Zhang Q, Larson D E, Shen D, McLellan M D, Lin L, Miller C A, Mardis E R, Ding L, Wilson R K. VarScan 2: somatic mutation and copy number alteration discovery in cancer by exome sequencing. Genome Res. 2012 March; 22(3):568-76. doi: 10.1101/gr.129684.111. Epub 2012 Feb. 2. PubMed PMID: 22300766; PubMed Central PMCID: PMC3290792.

Langmead B, Salzberg S L. Fast gapped-read alignment with Bowtie 2. Nat Methods. 2012 Mar. 4; 9(4):357-9. doi: 10.1038/nmeth.1923. PubMed PMID: 22388286; PubMed Central PMCID: PMC3322381.

Lawrence M S, Stojanov P, Mermel C H, et al, (2014) Discovery and saturation analysis of cancer genes across 21 tumour types. Nature 505:495-501. doi: 10.1038/nature12912

Lawrence M S, Stojanov P, Polak P, et al. (2013) Mutational heterogeneity in cancer and the search for new cancer-associated genes. Nature 499:214-218, doi: 10.1038/nature12213

Li H, Durbin R. Fast and accurate short read alignment with Burrows-Wheeler transform (2009) Bioinformatics. 2009 Jul. 15; 25(14):1754-60. doi: 10.10931bioinformatics/btp324.

Langmead B, Trapnell C, Pop M, Salzberg S L (2009) Ultrafast and memory-efficient alignment of short DNA sequences to the human genome. Genome Biol. 2009; 10(3):R25. doi: 10.1186/gb-2009-10-3425.

Liaw A, Wiener M (2002) Classification and Regression by randomForest. R News 2:18-22.

Ma X-J, Patel R, Wang X, et al. (2006) Molecular classification of human cancers using a 92-gene real-time quantitative polymerase chain reaction assay. Archives of pathology & laboratory medicine 130:465-473.

Mitelman F, Mertens F, Johansson B (1997) A breakpoint map of recurrent chromosomal rearrangements in human neoplasia. Nat Genet 15 Spec No:417-474. doi: 10.1038/ng0497supp-417

Pavlidis N, Pentheroudakis G (2012) Cancer of unknown primary site. Lancet 379:1428-1435. doi: 10.1016/S0140-6736(11)61178-1

Ramaswamy S, Tamayo P, Rifkin R, et al, (2001) Multiclass cancer diagnosis using tumor gene expression signatures. Proc Natl Acad Sci USA 98;15149-15154. doi: 10.1073/pnas.211566398

Rosenfeld N, Aharonov R, Meiri E, et al. (2008) MicroRNAs accurately identify cancer tissue origin. Nat Biotechnol 26:462-469, doi: 10.1038/nbt1392

1. A method for prediction of a specific type of cancer in a subject using an acquired bodily sample from said subject, said method comprising the steps of:
a) providing biological sequences derived from said bodily sample,
b) deriving the mutation status of specific genes that are mutated in cancer from said biological sequences compared to a normal sample,
c) calculating one or more of the following types of information i) to iii) from said biological sequences:
i) single base substitution frequency wherein the identity of the two bases flanking said substitution is not taken into account,
ii) single base substitution frequency in triplets of nucleotide bases, wherein the identity of the two bases flanking the substitution is taken into account,
iii) copy number variation (CNV) of genomic regions and/or genes compared to the copy number of the same regions and/or genes in a normal sample
d) calculating a classification score for the presence of each of a plurality of cancer types in said subject, wherein said classification score calculation is based on the mutation status derived from step b) in combination with the one or more of the information types i) to iii) being calculated in step c),
e) ranking the plurality of cancer types based on the classification score of step d), and
f) predicting the specific type of cancer in said subject based on the ranking of step e).

2) The method according to claim 1, wherein the method calculates said classification score based on a combination of the mutation status derived from step b) in combination with information types i) and/or ii) of step c), or based on a combination of the mutation status derived from step b) in combination with information type iii) of step c).

3) The method according to the preceding claims, wherein the method calculates said classification score based on a combination of the mutation status derived from step b) in combination with information type iii) and one or more of the information types i) and ii) of step c).

4) The method according to the preceding claims, wherein the method calculates said classification score based on a combination of the mutation status derived from step b) in combination with information types iii) and ii) of step c).

5) The method according to the preceding claims, wherein said biological sequence is a DNA and/or mRNA sequence obtained from said bodily sample.

6) The method according to the preceding claims, wherein both synonymous and non-synonymous mutations are used for deriving said mutation status, or wherein only non-synonymous mutations are used for deriving said mutation status.

7) The method according to the preceding claims, wherein the mutation status of step b) and/or information type iii) of step c) is based on mutation status or copy number variation of genes that are recurrently mutated in association with cancer, such as the set of genes encoding the sequences of SEQ ID NO: 1 to 231.

8) The method according to any of the preceding claims, wherein said method is computer-implemented and involves the use of at least one classifier or a plurality of classifiers that are based on a machine learning method, preferably selected from the group consisting of decision trees, random forests, stepwise additive logistic regression, artificial neural networks and support vector machines, and more preferably wherein the machine learning method is random forests.

9) The method according to any of the preceding claims, wherein the plurality of cancer types comprises or consists of at least the following types of cancer: breast, endometrium, kidney, large intestine, liver, lung, ovary, pancreas, prostate, and skin cancer.

10) The method according to any of the preceding claims, wherein the plurality of cancer types comprises or consists of at least the following types of cancer: breast, endometrium, kidney, large intestine, lung and ovary cancer.

11) The method according to any of the preceding claims, further comprising a step wherein a confidence score is calculated as the difference between the classification scores from the two highest ranking types of cancer.

12) The method according to any of the preceding claims, wherein the bodily sample is a bodily tissue sample such as a biopsy sample or a bodily fluid sample such as sample of blood, serum, plasma, urine, lymph fluid, sputum or bronchial washing fluid.

13) A computer program product having instructions which when executed by a computing device or system causes the computing device or system to carry out the method according to any one of claims 1 to 12.

14) A data-processing system having means for carrying out the method according to any one of claims 1 to 12.

15) A computer readable medium having stored thereon a computer program product according to claim 13.
